(19) United States
(12) Patent Application Publication
(10) Pub. No.: US 2024/0035065 A1
(43) Pub. Date: Feb. 1, 2024

(54) METHOD FOR MEASUREMENT IN BIOSENSORS

(71) Applicant: The University of North Carolina at Chapel Hill, Chapel Hill, NC (US)

(72) Inventors: Koji Sode, Chapel Hill, NC (US); Jeffrey Dick, Chapel Hill, NC (US); Nicole Walker, Chapel Hill, NC (US); David Probst, Chapel Hill, NC (US); Inyoung Lee, Chapel Hill, NC (US); Shouhei Takamatsu, Chapel Hill, NC (US)

(21) Appl. No.: 18/265,797

(22) PCT Filed: Dec. 7, 2021
(86) PCT No.: PCT/US2021/062189

§ 371 Date: Jun. 7, 2023

Related U.S. Application Data
(60) Provisional application No. 63/122,391, filed on Dec. 7, 2020.

(51) Int. Cl. C12Q 1/26; C12Q 1/00

(52) U.S. Cl. CPC C12Q 1/26; C12Q 1/005

(57) ABSTRACT

Devices and methods are disclosed for measuring a target substance concentration in a sample utilizing a biosensor.

### CROSS-REFERENCE TO RELATED APPLICATION

This application claims the benefit of and priority to U.S. Provisional Application No. 63/122,391, filed Dec. 7, 2020, which is incorporated by reference herein in its entirety.

### GOVERNMENT SUPPORT

This invention was made with government support under Grant No. GM138133 awarded by the National Institutes of Health. The government has certain rights in the invention.

### REFERENCE TO A SEQUENCE LISTING SUBMITTED AS A TEXT FILE VIA EFS WEB

The Sequence Listing written in file 570837_SEQ.txt is 1 kilobyte, was created on Nov. 26, 2021, and is hereby incorporated by reference.

### BACKGROUND

Many current electrochemical sensors, such as sensors for self-monitoring blood glucose, continuous glucose monitoring sensors, and other enzyme sensors used clinically, in food process monitoring, and in other analytical purposes, employ amperometric measurement of oxidation reactions of substrates catalyzed by oxidoreductases.

However, amperometric enzyme sensors have an inherent issues when it comes to downsizing the sensor size. The miniaturization limitation of the currently available enzyme sensors is due to the method of the amperometric measurement itself, i.e., the measurement current (i.e., oxidation of hydrogen peroxide or oxidation/reduction of an electron acceptor) as a function of time and the catalytic current depends on the surface area of the electrode.

There remains a need to develop methods and devices that enable an accurate, stable, and long-term quantitative measurement of the concentration of a target substance using a miniaturized biosensor.

### BRIEF SUMMARY

Devices and methods are provided for measuring a target substance concentration in a sample.

In one embodiment, a method of measuring a target substance concentration in a sample comprising: contacting the sample comprising the target substance with a biosensor which comprises an enzyme electrode comprising an oxidoreductase immobilized on the electrode and a reference electrode; measuring a time-dependent change of an open circuit potential between the enzyme electrode and reference electrode; and calculating the concentration of the target substance based on the time-dependent change of the open circuit potential. In one embodiment, a method of measuring a target substance concentration in a sample continuously. In embodiments, the biosensor further comprises a counter electrode.

In another embodiment, a biosensor for measuring a concentration of a target substance in a sample comprising an enzyme electrode comprising an oxidoreductase immobilized on the electrode and a reference electrode. In one embodiment, the reference electrode is a leakless reference electrode comprising a sealed platinum wire. In embodiments, the biosensor further comprises a counter electrode.

### BRIEF DESCRIPTION OF THE SEVERAL VIEWS OF THE DRAWING(S)

Having thus described the invention in general terms, reference will now be made to the accompanying drawings, which are not necessarily drawn to scale.

FIG. 1 illustrates electrochemical evaluations of the OCP change on the PES-modified DAAOx electrode employing electrochemical OCP measurement principle (A) OCP evaluation of the DAAOx immobilized electrode which was modified by arPES in 100 mM PPB (pH 8.0), followed by the addition of 0.01, 0.02, 0.05, 0.1, 0.25, 0.5, 1, 2.5, 5, 10, 20, and 30 mM D-serine at the timing indicated by bottom blue arrows, and +100 mV (vs. Ag/AgCl) was applied at the timing indicated by upper gray arrows. (B) The OCP change at each D-serine concentration with respect to the difference time (δ time) that adjusted 0 sec to immediately after applying a potential at 100 mV vs. Ag/AgCl.

FIG. 2 illustrates time dependency of the changing rate of OCP (dOCP/dt) on OCP monitoring using PES-DAAOx electrode with condition including 0, 0.01, 0.02, 0.05, 0.1, 0.25, 0.5, 1, 2.5, 5, 10, 20, and 30 mM D-serine. The lines of 10, 20 and 30 mM D-serine condition were overlapped.

FIG. 3 illustrates calibration curves of D-serine sensing using PES-DAAOx electrode based on OCP measurement principle Calibration curve of OCP changing rate (dOCP/dt) at δ time=0 sec toward D-serine concentration representing kinetic characteristics of OCP measurement. Lower concentration range was enlarged and shown in inset. This experiment was performed using three electrodes and the results were displayed as a triplicated (n=3) with standard deviation error bars.

FIG. 4 illustrates electrochemical evaluations of the OCP change on the PES-modified LOx electrode employing electrochemical OCP measurement principle (A) Time course of OCP evaluation using the PES-modified LOx immobilized electrode in 100 mMPPB (pH 7.0), followed by the addition of 0.05, 0.1, 0.3, 0.5, 1, 2, 5, 10 and 20 mM L-lactate at the bottom blue arrows, and +100 mV (vs. Ag/AgCl) potential application at the timing indicated by upper gray arrows. (B) The OCP change at each L-lactate concentration with respect to the difference time (δ time) that adjusted 0 sec to immediately after applying a potential at 100 mV vs. Ag/AgCl.

FIG. 5 illustrates time dependency of the changing rate of OCP (dOCP/dt) on OCP monitoring using PES-LOx electrode with condition including 0.05, 0.1, 0.3, 0.5, 1, 2, 5, 10 and 20 mM lactate. The lines of 2, 5, 10 and 20 mM lactate condition were overlapped.

FIG. 6 illustrates calibration curves of lactate sensing using PES-LOx electrode based on OCP measurement principle (A) The ΔOCP value that is the difference of steady-state OCP value from minimum substrate concentration (0.01 mM L-lactate in this experiment) to each substrate concentration were plotted against to the L-lactate concentration with logarithmic scale. (B) Calibration curve of OCP changing rate (dOCP/dt) at δ time=0 sec toward lactate concentration representing kinetic characteristics of OCP measurement using PES-LOx electrode. Lower concentration range was enlarged and shown in inset. This experiment was performed using three electrodes and the results were displayed as a triplicated (n=3) with standard deviation error bars.

FIG. 7 illustrates electrochemical evaluations of the OCP change on the PES-modified GDH electrode employing electrochemical OCP measurement principle (A) OCP evaluation of the PES-modified GDH immobilized electrode in 100 mM PPB (pH 7.0), followed by the addition of 0.1, 1, 3, 5, 10, 15, and 20 mM glucose at the bottom blue arrows and +100 mV (vs. Ag/AgCl) potential application at the timing indicated by upper gray arrows. (B) The OCP change at each lactate concentration with respect to the difference time (δ time) that adjusted 0 sec to immediately after applying a potential at 100 mV vs. Ag/AgCl.

FIG. 8 illustrates time dependency of the changing rate of OCP (dOCP/dt) on OCP monitoring using arPES-GDH electrode with condition including 0.1, 1, 3, 5, 10, 15 and 20 mM glucose. The lines of 15 and 20 mM glucose condition were overlapped.

FIG. 9 illustrates calibration curves of lactate sensing using PES-GDH electrode based on OCP measurement principle (A) The ΔOCP value that is the difference of steady-state OCP value from minimum substrate concentration (0.1 mM glucose in this experiment) to each substrate concentration were plotted against to the lactate concentration with logarithmic. (B) Calibration curve of OCP changing rate (dOCP/dt) at δ time=0 sec toward lactate concentration representing kinetic characteristics of OCP measurement using PES-GDH electrode. Lower concentration range was enlarged and shown in inset. This experiment was performed using three electrodes and the results were displayed as a triplicated (n=3) with standard deviation error bars.

FIG. 10 illustrates a representative time course of OCP and dOCP/dt. Sensor response toward glucose using a direct electron transfer-type FADGDH (DET-FADGDH) electrode based on OCP measurement principle (A) Time course of sensor response upon potential application (cyan arrows) and glucose additions (red arrows). (B) Time course of sensor response (OCP) after potential application.

FIG. 11 illustrates a calibration curve of dOCP/dt. Calibration curves of glucose sensing using Direct electron transfer-type FADGDH (DET-FADGDH) electrode based on OCP measurement principle (A) Calibration curve of OCP changing rate (dOCP/dt) at δ time=0 sec toward glucose concentration representing kinetic characteristics of OCP measurement using DET-FADGDH electrode. This experiment was performed by carrying out three independently prepared electrodes (n=3) with standard deviation error bars. (B) Calibration curve of ΔdOCP/dt was normalized by subtracting dOCP/dt value at the 0 mM glucose.

FIG. 12 illustrates a calibration curve of dOCP/dt. Calibration curves of glucose sensing using a direct electron transfer-type FADGDH (DET-FADGDH) electrode based on OCP measurement principle (A) Calibration curve of OCP changing rate (dOCP/dt) at δ time=0 sec toward glucose concentration representing kinetic characteristics of OCP measurement using DET-FADGDH electrode. This experiment was performed by carrying out three times repeated measurements and the results were displayed as a triplicated (n=3) with standard deviation error bars. (B) Calibration curve of ΔdOCP/dt was normalized by subtracting dOCP/dt value at the 0 mM glucose.

FIG. 13 illustrates the repeatability of dOCP/dt during continuous operation. Repeatability evaluation of OCP measurement using the DET-GDH immobilized electrode (A) Time course of OCP measurement using the DET-FADGDH immobilized electrode in 100 mM PPB (pH 7.0) including 20 mM glucose with 10-times potential application of +100 mV (vs. Ag/AgCl) at the timing indicated by gray arrows. (B) The OCP change at each potential application with respect to the difference time (δ time) that adjusted 0 sec to immediately after applying a potential at 100 mV (vs. Ag/AgCl). (C) Time dependency of the changing rate of OCP(dOCP/dt) on DET-FADGDH electrode. All lines were overlapped. (D) The repeatability of OCP measurement for 10-times using DET-GSH electrode.

FIG. 14 illustrates OCP data of a 10 micrometer electrode. Electrochemical evaluations of the OCP change on the micro-sized DET-FADGDH electrode (electrode surface area: 0.785 μm²) employing electrochemical OCP measurement principle (A) OCP evaluation of the micro-sized DET-FADGDH electrode, followed by the addition of 1, 3, 5, 10, 15 and 20 mM glucose at the timing indicated by bottom red arrows. (B) The OCP value were plotted against to glucose concentration with logarithmic. (C) The ΔOCP value that is a difference of steady-state OCP value from minimum substrate concentration (1 mM glucose in this experiment) to each substrate concentration were plotted against to the glucose concentration with logarithmic

FIG. 15 illustrates dOCP/dt change of a 10 micrometer electrode. Electrochemical evaluation of the OCP change on the micro-sized DET-FADGDH electrode (electrode surface area: 0.785 μm²) employing electrochemical OCP measurement principle (A) The OCP change at each potential application with respect to the difference time (δ time) that adjusted 0 sec to immediately after applying a potential at 100 mV (vs. Ag/AgCl). (B) Time dependency of the changing rate of OCP(dOCP/dt) on micro-sized DET-FADGDH electrode (C) Enlarged figure (B).

FIG. 16 illustrates dOCP/dt change of a 10 micrometer electrode. Calibration curves of glucose sensing using micro-sized DET-FADGDH electrode (electrode surface area: 0.785 μm²) based on OCP measurement principle (A) Calibration curve of OCP changing rate (dOCP/dt) at δ time=0 sec toward glucose concentration representing kinetic characteristics of OCP measurement using micro-sized DET-FADGDH electrode. (B) Calibration curve of ΔdOCP/dt was normalized by subtracting dOCP/dt value at 0 mM glucose.

FIG. 17 illustrates an experiment for a bipolar reference electrode. A commercial Ag/AgCl reference electrode is placed in vial 1, containing 1 M KCl, and a gold macroelectrode is placed in vial 2, containing 1 mM ferricyanide and 1 mM ferrocyanide in 250 mM KCl. A platinum wire connects vials 1 and 2, allowing for the transfer of electrons between the two vials. A potentiostat measures the open circuit potential between the working and reference electrodes.

FIG. 18 illustrates the open circuit potential of (blue) the gold macroelectrode (r=1 mm) and commercial Ag/AgCl reference electrode in a solution of 1 mM ferricyanide and 1 mM ferrocyanide in 250 mM KCl, using a gold macroelectrode (r=1 mm) for 1 hour and (green) the commercial Ag/AgCl reference electrode in one vial of 1 M KCl with the gold macroelectrode in a second vial containing 1 mM ferricyanide and 1 mM ferrocyanide in 250 mM KCl and a platinum wire acting as a bridge between the two vials.

FIG. 19 illustrates a schematic of a bipolar reference electrode. A platinum wire (d=0.025 mm) is sealed in the tip of a borosilicate capillary tube (OD=2 mm, ID=1.16 mm). A 1 M KCl filling solution is added in via syringe, then the Ag/AgCl wire (d=0.025 mm) is placed into the back end of the tube. Copper tape on the silver portion of the Ag/AgCl wire acts as the place of contact for the reference electrode.

FIG. 20 illustrates the open circuit potential of 3 commercial Ag/AgCl reference electrodes in a solution of 1 mM ferricyanide and 1 mM ferrocyanide in 250 mM KCl, using a gold macroelectrode (r=1 mm) as the working electrode.

FIG. 21 illustrates the open circuit potential of 10 bipolar reference electrodes in a solution of 1 mM ferricyanide and 1 mM ferrocyanide in 250 mM KCl, using a gold macroelectrode (r=1 mm) as the working electrode.

FIG. 22 illustrates the open circuit potential of 4 miniature bipolar (miniBP) reference electrodes in a solution of 1 mM ferricyanide and 1 mM ferrocyanide in 250 mM KCl, using a gold macroelectrode (r=1 mm) as the working electrode.

FIG. 23 illustrates a comparison of the average open circuit potential of the commercial Ag/AgCl reference electrodes, the bipolar reference electrodes, and the miniature bipolar electrodes in 1 mM ferrocyanide in 250 mM KCl, using a gold macroelectrode as the working electrode.

FIG. 24 illustrates the open circuit potentiometry response of a of GOx-chitosan platinum macroelectrode as 1 μM to 3 mM glucose is spiked into a solution of DPBS (n=3), using a bipolar reference electrode.

FIG. 25 illustrates the open circuit potentiometry response of a of GOx-chitosan platinum macroelectrode as 1 μM to 3 mM glucose is spiked into a solution of DPBS (n=3, each), using a (green) bipolar reference electrode and (blue) commercial Ag/AgCl reference electrode. The two sets of calibration curves are statistically insignificantly different.

FIG. 26 illustrates an experiment testing for ion leakage out of the tip of the bipolar reference electrode, one was filled with 1 mM ferricyanide and 1 mM ferrocyanide in 250 mM KCl and stored in a vial of 1 M KCl. Cyclic voltammograms were taken of the solution using a commercial Ag/AgCl reference electrode, a gold macroelectrode, and a glassy carbon counter electrode on day 0 (before the bipolar reference electrode was placed in solution) and periodically for the next 18 days. Lack of peaks near 0.16 V and 0.36V indicates that none of the ferri/ferrocyanide leaked out of the tip of the bipolar reference electrode over 18 days.

FIG. 27 illustrates an experiment to test for ion leakage out of the tip of the bipolar reference electrode, one was filled with 1 mM ferricyanide and 1 mM ferrocyanide in 250 mM KCl and stored in a vial of 1 M KCl. Cyclic voltammograms (CV) were taken of the solution using a commercial Ag/AgCl reference electrode on day 0 (before the bipolar reference electrode was placed in solution) and periodically for the next 18 days. Another cyclic voltammogram of 1 mM ferricyanide and 1 mM ferrocyanide in 1 M KCl shows where the peaks would appear if the ferri/ferrocyanide was leaking out into the vial of 1 M KCl.

FIG. 28 illustrates an experiment determining what the effects of temperature were on the bipolar reference electrode, a solution of 1 mM ferricyanide and 1 mM ferrocyanide in 250 mM KCl and stored in a vial of 1 M KCl. The solution was heated on a hot plate to 11° C., 15° C., 21° C., 30° C., and 40° C., and the open circuit potential was measured for 30 minutes using a gold macroelectrode and either a commercial reference electrode or one of two bipolar reference electrodes. There is a similar change in open circuit potential as temperature increases using the commercial and the bipolar reference electrodes.

FIG. 29 illustrates an experiment showing that the bipolar reference electrode works in organic solution, the open circuit potential was measured for 30 minutes in a solution of 1 mM ferrocene in a solution of 100 mM tetrabutylammonium perchlorate in acetonitrile for 30 minutes, using a gold macroelectrode and either a commercial reference electrode or one of three bipolar reference electrodes.

FIG. 30 illustrates a chromatographic profile of the crude E. coli LOBSTR containing the N-terminal His-tagged D-amino acid oxidases (DAAOxs) (A) wild-type (WT) and (B) Gly52Val (G52V) mutant, separated with the Ni²⁺ affinity column. The starting points for each sample loading, washing, and elution are indicated by black arrows. The protein absorbance at 280 nm (black) and also cofactor flavin adenine dinucleotide (FAD)-derived absorbance at 450 nm (yellow), the 0 to 400 mM linear gradient for imidazole concentration (orange) were represented. The peaks where an increase in absorbance of both A280 and A450 was observed were collected and used as the purified enzyme.

FIG. 31 illustrates an SDS-PAGE profile of the purified D-amino acid oxidases (DAAOxs) obtained by Ni²⁺ affinity column chromatography. Protein marker, purified DAAOx wild-type (WT) and Gly52Val (G52V) were applied to the gel sequentially. The sizes of some bands in the protein marker are shown to the left of the PAGE gel image. Arrows show the indicated overexpressed His-tagged DAAOx (near to 41.8 kDa).

FIG. 32 illustrates an enzyme activity assay of wild-type (WT) and Gly52Val (G52V) mutant of D-amino acid oxidase (DAAOx). (A) DAAOx has a reductive half-reaction, which involves the reduction of the cofactor flavin adenine dinucleotide (FAD) with oxidation of substrate, and an oxidative half-reaction, which involves the oxidation of FAD and the transfer of electrons to the electron acceptor. The reaction that uses oxygen as an electron acceptor is called oxidase activity, and the reaction that uses artificial synthetic electron acceptors (mediators) instead of oxygen is called dehydrogenase activity. (B) The oxidase activity using D-serine as a substrate was measured by using a dye-coloration reaction associated with hydrogen peroxide produced. Closed circles indicate WT and open circle indicate G52V. (C) The dehydrogenase activity using d-serine as a substrate is measured using the artificial synthetic electron acceptors (mediators), PMS and DCIP, with the fading reaction of DCIP as an indicator. Closed square indicate WT and open squares indicate G52V. All measurements were carried out in 100 mM potassium phosphate buffer (pH 8.0) at 37° C., and performed with triplicated (n=3). Error bars at each point indicate the standard deviation of each measurement.

FIG. 33 illustrates a representative cyclic voltammograms of electrodes immobilized Gly52Val (G52V) mutant of D-amino acid oxidase (DAAOx). In each, phenazine ethosulfate (PES)-modified electrode was shown as solid red line, and unmodified one shown as dashed gray line. The cyclic voltammetry was performed with scan rate at 100 mV/s in 100 mM potassium phosphate buffer (pH 8.0) at room temperature (around 25° C.) with triplicated (n=3). The potentiostat (VSP) was used in this experiment.

FIG. 34 illustrates an amperogram using electrodes immobilized with D-amino acid oxidase (DAAOx) Gly52Val (G52V) mutant modified with amine-reactive phenazine ethosulfate. (A) Amperometry by applying potential as 0 mV vs. Ag/AgCl while 0.05, 0.1, 0.25, 0.5, 1, 2.5, 5, 10, 20, 30 mM D-serine was added to the measurement buffer and the change in current was observed. (B) Calibration curve of the steady-state current plotted against D-serine. From Eadie-Hofstee plot, maximum steady current (Iₘₐₓ) and apparent affinity (Kₘ₍ₐₚₚ₎) against D-serine were calculated as 24.1 nA and 2.3 mM, respectively. A potentiostat (SP-150) was used for the experiments.

FIG. 35 illustrates representative transient potentiograms and calibration curves in open circuit potential (OCP) measurements using electrodes with immobilized D-amino acid oxidase (DAAOx) Gly52Val (G52V), and modified by amine-reactive phenazine ethosulfate (arPES). (A) OCP measurements were performed for 300 s following the initial 0.1 s application of a 100 mV vs. Ag/AgCl. The OCP was plotted against the time after the potential application (δtime). The measurements were performed in the absence of d-serine and in the presence of 0.02, 0.05, 0.2, 0.5, 2, 5, 10, 20, and 30 mM. (B) From FIG. 35A, the ΔOCP was calculated by subtracting the OCP value in the absence of D-serine from each steady-state OCP at 100 s (circles), 200 s (squares), and 300 s (diamonds) after application of the potential, and plotted against the d-serine concentration, respectively. An inset showed an enlarged calibration between 0 to 0.5 mM d-serine. (C) The rate of change in OCP (dOCP/dt) was determined by time differentiation of the change in OCP at each time and plotted against δ time. Inset shown an enlarged view of the first 2 s of measurement which are framed in red in FIG. 35C. (D) The calibration curve using dOCP/dt as a read-out value was obtained by plotting the dOCP/dt value at 0.1 s (circle), 0.5 s (square), 2 s (diamond), 5 s (triangle) and 10 s (cross) against D-serine concentration, respectively. (E) Potentiograms converted to square roots with respect to δtime from FIG. 35A. (F) Enlarged view of the first 2 s of measurement which are framed in red in FIG. 35F. (G) Time course of dOCP/d√t for initial 2 s at varied concertation of D-serine. (H) Calibration curve of averaged OCP change rate for square root of time (dOCP/d√t) which plotted against the concentration of D-serine. All measurements were performed in a Faraday cage in 100 mM potassium phosphate buffer (pH 8.0), stirred at 250 rpm with a magnetic stirrer, at room temperature (around 25° C.). Experiments at same condition have been performed on three independent electrodes (n=3), and the standard deviation of each measurement was indicated by an error bar for FIGS. 35B, 35F and 35H. A potentiostat (SP-150) was used for this experiment.

FIG. 36 illustrates an enlarged view of lower D-serine concentration from FIG. 35. All measurements were performed on three independent electrodes (n=3) and the standard deviation of each measurement is indicated by error bars. A potentiostat (SP-150) was used for the experiments.

FIG. 37 illustrates representative potentiograms of continuous open circuit potential (OCP) measurements using an electrode with immobilized D-amino acid oxidase (DAAOx) Gly52Val (G52V) mutant, and modified by amine-reactive phenazine ethosulfate (arPES). (A) Continuous OCP measurement was performed by repeating two steps: a potential application of 100 mV vs. Ag/AgCl for 0.1 s and an OCP measurement for 1.9 s. Throughout the operation, D-serine at final concentrations of 0.02, 0.05, 0.2, 0.5, 2, 5, 10, 20, and 30 mM was added at 30 s intervals (timing indicated by black arrows). (B) Enlarged view between 170 and 200 s of the measurement in FIG. 37A, where D-serine was added at 180 s to reach a final concentration of 2 mM, with a corresponding marked change. (C) Represented OCP change at each concentration of D-serine.

FIG. 38 illustrates calibration curves of open circuit potential (OCP) change rate against square root of time (dOCP/d√t) for D-serine at continuous OCP measurement using electrodes immobilized with D-amino acid oxidase (DAAOx) Gly52Val (G52V) and wild-type (WT). (A) Calibration curves of the DAAOx G52V-immobilized electrodes during continuous OCP measurements with and without amine-reactive phenazine ethosulfate (PES) modification and under ambient or argon gas atmosphere conditions. The results of ambient atmosphere conditions of PES-modified DAAOx G52V electrode (red closed circles), argon gas atmospheric conditions of PES-modified DAAOx G52V electrode (red open squares), ambient atmosphere conditions of PES-unmodified DAAOx G52V electrode (black closed circles), and argon gas atmosphere conditions of PES-unmodified DAAOx G52V electrode (black open squares) are plotted against d-serine concentration. Each point of the DAAOx G52V and DAAOx G52V-PES electrodes under ambient and argon gas atmosphere almost overlapped. (B) The results of ambient atmosphere conditions of PES-modified DAAOx WT electrode (red closed triangle), argon gas atmospheric conditions of PES-modified DAAOx WT electrode (red open triangle), ambient atmosphere conditions of PES-unmodified DAAOx WT electrode (black closed diamond), and argon gas atmosphere conditions of PES-unmodified DAAOx WT electrode (black open diamond) are plotted against D-serine concentration.

FIG. 39 illustrates representative cyclic voltammograms of electrodes immobilized with wild-type (WT) of D-amino acid oxidase (DAAOx). A phenazine ethosulfate (PES)-modified electrode was shown as solid red line, and unmodified one shown as dashed gray line. The cyclic voltammetry was performed with a scan rate of 100 mV/s in 100 mM potassium phosphate buffer (pH 8.0) at room temperature (around 25° C.) in triplicate (n=3). The potentiostat (VSP) was used in this experiment.

FIG. 40 illustrates continuous open circuit potential (OCP) measurements under ambient and argon gas atmosphere using electrodes immobilized with representative D-amino acid oxidase (DAAOx) wild-type (WT) modified and unmodified with amine-reactive phenazine ethosulfate (PES). The OCP was measured continuously by repeating the cycle of applying a potential of 100 mV vs. Ag/AgCl for 0.1 s and then measuring OCP for 1.9 s. The OCP responses at sufficient time after the addition of each concentration of D-serine were extracted and plotted against the square root of time. (A) OCP responses of PES-modified WT electrode to D-serine concentration under ambient atmosphere. (B) OCP responses of PES-modified WT electrode to D-serine under argon gas atmosphere. (C) OCP responses of PES unmodified WT electrode to D-serine concentration under ambient atmosphere. (D) OCP responses of PES unmodified WT electrode to D-serine concentration under argon gas atmosphere. The measurements were performed with three independent electrodes (n=3) under each condition, and representative results were described. A potentiostat (SP-150) was used for the experiments.

FIG. 41 illustrates specificity evaluation in continuous open circuit potential (OCP) measurement using an electrode immobilized with amine-reactive phenazine ethosulfate-modified D-amino acid oxidase (DAAOx) Gly52Val (G52V). During continuous OCP measurements, L-serine (white circle), D-aspartate (gray square) or L-glutamate (black triangle) at final concentrations of 0.01, 0.05, 0.2, 0.5, 2, 5, 10, 20, and 30 mM were added, and the OCP change rate against square root of time (dOCP/d√t) at each ligand concentrations were plotted. All measurements were performed on three independent electrodes (n=3) and the standard deviation of each measurement is indicated by error bars. A potentiostat (SP-150) was used for the experiments.

FIG. 42 illustrates evaluation of the effect of applied potential on continuous open circuit potential (OCP) measurement using an electrode immobilized with amine-reactive phenazine ethosulfate-modified D-amino acid oxidase (DAAOx) Gly52Val (G52V) mutant. The D-serine-dependent OCP change rates against square root of time (dOCP/d√t) were evaluated when the applied potentials for continuous OCP measurements were 150 mV (circle), 50 mV (square), 0 mV (diamond) and −50 mV (triangle) vs. Ag/AgCl. All measurements were performed at three independent electrodes (n=3), and the standard deviation of each measurement was indicated by error bars. A potentiostat (SP-150) was used for the experiments.

FIG. 43 illustrates the evaluation of D-serine levels in artificial human cerebrospinal fluid (aCSF) using OCP measurements, with and without albumin, using the PES-modified DAAOx G52V electrode. (A) OCP response to sequential addition of D-serine in aCSF. (B) dOCP/d√t responses to additions of D-serine in aCSF. (C) OCP response to sequential addition of D-serine in aCSF with HSA. (D) dOCP/d√t responses to additions of D-serine in aCSF with HSA.

FIG. 44 illustrates calibration curves for ΔOCP and dOCP/dt in aCSF for the experiment discussed with respect to FIG. 43. (A) Calibration curves of ΔOCP for D-serine additions in aCSF. (B) Calibration curves of dOCP/dt for D-serine additions in aCSF. (C) Calibration curves of ΔOCP for D-serine additions in aCSF with HSA. (D) Calibration curves of dOCP/dt for D-serine additions in aCSF with HSA.

FIG. 45 illustrates calibration curves for dOCP/d√t in aCSF for the experiment discussed with respect to FIG. 43. (A) Calibration curves of dOCP/d√t for D-serine additions in aCSF. (B) Calibration curves of dOCP/d√t for D-serine additions in aCSF.

### DETAILED DESCRIPTION

The presently disclosed subject matter will now be described more fully hereinafter. However, many modifications and other embodiments of the presently disclosed subject matter set forth herein will come to mind to one skilled in the art to which the presently disclosed subject matter pertains having the benefit of the teachings presented in the foregoing descriptions. Therefore, it is to be understood that the presently disclosed subject matter is not to be limited to the specific embodiments disclosed and that modifications and other embodiments are intended to be included within the scope of the appended claims. In other words, the subject matter described herein covers all alternatives, modifications, and equivalents. In the event that one or more of the incorporated literature, patents, and similar materials differs from or contradicts this application, including but not limited to defined terms, term usage, described techniques, or the like, this application controls. Unless otherwise defined, all technical and scientific terms used herein have the same meaning as commonly understood by one of ordinary skill in this field. All publications, patent applications, patents, and other references mentioned herein are incorporated by reference in their entirety.

### I. Overview

Biosensors are described as analytical devices incorporating a biological material, a biologically derived material or a biomimic intimately associated with or integrated within a physicochemical transducer or transducing microsystem, which may be optical, electrochemical, thermometric, piezoelectric, magnetic or micromechanical (Turner et al., 1987; Turner, 1989 in Biosensors and Bioelectronics). The most representative and industrially important sensors are the electrochemical enzyme sensors for glucose. Theses electrochemical sensors employ amperometric measurements of oxidation reactions of substrates catalyzed by oxidoreductases. To detect these redox-reactions, electrical current is monitored by measuring the consumed electron acceptor (e.g. decreased oxygen concentration in oxidase reaction) or measuring reduced electron acceptors caused by the oxidation of substrate. Reduced electron acceptors, such as hydrogen peroxide as the reduced form of oxygen, or reduced artificial synthetic electron acceptors or so called mediators, will then be oxidized on electrode to monitor current. Alternatively, by employing some specific enzymes capable of transferring electrons formed by the oxidation of substrate directly to the electrode, i.e., direct electron transfer-type enzymes, or DET-enzymes, amperometric enzyme sensors are constructed by oxidizing DET-enzyme directly on electrode.

However, amperometric enzyme sensors have an inherent issue to downsizing the sensor size. The limitation of the size of the current available enzyme sensors is due to the method of the amperometric measurement itself, the measurement current (i.e., oxidation of hydrogen peroxide or oxidation/reduction of an electron acceptor) as function of time, and the catalytic current depends on the surface area of the electrode. The challenge to downsizing electrochemical sensors employing amperometry is that the current (signal proportional to electrode size) also decreases and would be indistinguishable from the noise of background current.

In contrast, it has now been found that open circuit potential (OCP) applied to enzyme sensors provides a practical solution to downsizing the sensor. The application of OCP to measure enzyme turnover kinetics was previously reported. A theoretical model incorporating enzymatic rate expressions into the Nernst equation was derived to explain the observed potential transients. The result obtained by the amperometric technique depended on the size of the electrode whereas the potentiometric technique was shown to be independent of the electrode size. Additionally, glucose biosensing principles based on the third generation principle that employs direct electron transfer (DET)-type glucose dehydrogenases (GDH) have been reported. Recently, a study of a third generation enzyme sensor based on open circuit potential (OCP) measurement using direct electron transfer FADGDH was reported. When the circuit is opened, minimal current (10-15 amperes or less) flows in the circuit; therefore, there is no appreciable current that can flow in the circuit; therefore, there is no appreciable current, and no application of a voltage is required for detection. Unlike the amperometric glucose sensor, glucose sensors employing OCP-based method generate a linear potentiometric response, correlating to a logarithmic scale of glucose concentration. However, the period to reach steady state potential takes more than 10 seconds—minute. In addition, current users of enzyme sensors require sensor signal linearity correlating to substrate concentration directly, not to a logarithmic scale, which is the theoretically inherent issue of OCP-based enzyme sensors.

Provided herein are methods and devices for measuring a time-dependent change of an open circuit potential and calculating a concentration of a target substance based on the time-dependent change of the open circuit potential. Based on the methods and devices provided herein, measurement of D-serine, lactate and glucose within 1 second by monitoring dOCP/dt with high reproducibility and accuracy can be achieved. Monitoring of glucose using a 10 μm electrode was also demonstrated based on dOCP/dt monitoring within 1 second.

These features as described herein show that enzyme sensors monitoring dOCP/dt have significant advantage compared with amperometric sensors and previously reported OCP based sensors.

### II. Devices

### Biosensors

In embodiments, described herein is a biosensor for measuring the concentration of a target substance in a sample. In embodiments, the biosensor comprises an enzyme electrode comprising an oxidoreductase immobilized on the electrode and a reference electrode.

In one embodiment, the working electrode can be inserted into buffer together with a counter electrode (such as a Pt electrode) and a reference electrode (such as an Ag/AgCl electrode), and kept at a predetermined temperature. A predetermined voltage can be applied to the working electrode, and then the sample is added and increased value in electric current is measured.

In embodiments, the biosensor is used in vivo. In embodiments, the biosensor is used in vivo inside of a cell of a subject. In embodiments, the biosensor is miniature, i.e., less than about 100 μm, and fits in a cell in vivo.

In embodiments, the target substance is selected from the group consisting of D-serine, lactate, glucose, glycated proteins, glycated amino acid, hydrogen peroxide, cholesterol, glycerol, glycerol-3-phosphate, fructose, urate, ethanol, galactose, 1,5-anhydro-D-glucitol, NAD(P)H, dopamine, 3-hydroxybutyrate, Levodopa (L-DOPA), L-glutamate, L-glutamine, sarcosine, creatine, and creatinine. In embodiments, the target substance is selected from the group consisting of D-serine, lactate, glucose, glycated proteins, glycated amino acid, hydrogen peroxide, cholesterol, glycerol, glycerol-3-phosphate, fructose, urate, ethanol, galactose, 1,5-anhydro-D-glucitol, NAD(P)H, dopamine, 3-hydroxybutyrate, and Levodopa (L-DOPA).

In embodiments, the biosensor further comprises a counter electrode. In embodiments, the biosensor comprises a counter electrode but it is switched off. In embodiments, a single electrode can function both as a counter electrode and a reference electrode. For example, a silver/silver chloride electrode or a calomel electrode may be used as the counter electrode, which can also function as a reference electrode.

The counter electrode is not limited as long as it can be generally used as a counter electrode for a biosensor. Examples of the counter electrodes include a carbon electrode prepared in the form of a film by screen printing, a metal electrode prepared in the form of a film by physical vapor deposition (PVD, for example, sputtering) or chemical vapor deposition (CVD), and a silver/silver chloride electrode prepared in the form of a film by screen printing. In embodiments, the counter electrode is made of conductive materials such as gold, palladium, platinum or carbon. In embodiments, the counter electrode is made of semi-conductor materials.

The term “in vitro” refers to artificial environments and to processes or reactions that occur within an artificial environment (e.g., a test tube).

The term “in vivo” refers to natural environments (e.g., a cell or organism or body) and to processes or reactions that occur within a natural environment.

### Working Electrodes

In another embodiment, the working electrode described herein is an enzyme electrode. In embodiments, an enzyme electrode comprising an oxidoreductase immobilized on the electrode. In embodiments, the oxidoreductase is immobilized on the electrode by cross-linking, encapsulating into a macromolecular matrix, coating with a dialysis membrane, optical cross-linking polymer, electroconductive polymer, oxidation-reduction polymer, and any combination thereof. In one embodiment, the oxidoreductase is immobilized on the working electrode together with an electron mediator such as potassium ferricyanide, ferrocene, osmium derivative, or phenazine methosulfate in a macromolecular matrix by means of adsorption or covalent bond to prepare a working electrode.

Oxidoreductases or redox-enzymes being used for the sensors are, for example, enzymes capable of direct electron transfer with electrode, such as redox enzymes harboring flavin adenine dinucleotide (FAD) or flavin mononucleotide (FMN) as cofactors. In embodiments, the oxidoreductase is selected from the group consisting of oxidases, dehydrogenases, monooxigenases and dioxygenases. In another embodiment, the oxidoreductase is selected from the group consisting of glucose dehydrogenase, glucose oxidase, lactate oxidase, lactate dehydrogenase, D-amino acid oxidase, fructosyl amino acid/peptide oxidases, peroxidase, cholesterol oxidase, glycerol-3-phosphate oxidase, cellobiose dehydrogenase, and fructose dehydrogenase, uricase, alcohol oxidase, alcohol dehydrogenase, galactose oxidase, galactose dehydrogenase, pyranose oxidase, pyranose dehydrogenase, glucose-3-dehydrogenase, diaphorase, thyrosinase, 3-hydroxybutyrate dehydrogenase, amine oxidase, monoamine oxidase, polyamine oxidase, dopamine β-monooxygenase, 4,5-DOPA dioxygenase extradiol, glutamate oxidase, and sarcosine oxidase. In another embodiment, the oxidoreductase is selected from the group consisting of glucose dehydrogenase, glucose oxidase, lactate oxidase, lactate dehydrogenase, D-amino acid oxidase, fructosyl amino acid/peptide oxidases, peroxidase, cholesterol oxidase, glycerol-3-phosphate oxidase, cellobiose dehydrogenase, and fructose dehydrogenase, uricase, alcohol oxidase, alcohol dehydrogenase, galactose oxidase, galactose dehydrogenase, pyranose oxidase, pyranose dehydrogenase, glucose-3-dehydrogenase, diaphorase, thyrosinase, 3-hydroxybutyrate dehydrogenase, amine oxidase, monoamine oxidase, polyamine oxidase, dopamine β-monooxygenase, and 4,5-DOPA dioxygenase extradiol.

In embodiments, the oxidoreductase is an engineered oxidoreductase. In embodiments, the engineered oxidoreductase is a fusion enzyme. In embodiments, the fusion enzyme comprises a flavin, e.g., FAD or FMN without heme domain or heme subunit or heme, e.g., heme b or heme c. In embodiments, the oxidoreductase is an electron mediator modified redox enzyme, which are with or without heme domain or heme subunit.

In embodiments, oxidoreductase is an oxidoreductase capable of direct transfer of electrons with the enzyme electrode. In embodiments, the oxidoreductase is an oxidoreductase containing an electron transfer subunit or an electron transfer domain. In embodiments, the electron transfer subunit or the electron transfer domain contains heme.

In embodiments, the working electrode is made of conductive materials such as gold, palladium, platinum, or carbon.

In embodiments, the working electrode is a screen printed carbon electrode, a planar gold electrode, or an interdigitated electrode array.

In embodiments, the working electrode described herein is miniature, e.g., less than about 100 μm. In one embodiment, the working electrode is less than about 2 mm, less than about 1 mm, or less than about 0.5 mm in diameter. In embodiments, the working electrode fits in a cell in vivo. In embodiments, the working electrode is less than about 100 μm, less than about 50 μm, less than about 25 μm, less than about 10 μm, or less than about 1 μm in diameter. In embodiments, the working electrode is about 1 μm to about 100 μm, about 10 μm to about 100 μm, about 25 μm to about 100 μm, about 50 μm to about 100 μm, about 75 μm to about 100 μm, about 1 μm to about 75 μm, about 1 μm to about 50 μm, about 1 μm to about 25 μm, or about 1 μm to about 10 μm in diameter.

### Reference Electrodes

In one embodiment, described herein are leakless reference electrodes. In another embodiment, the reference electrode as described herein is a leakless, non-porous electrode. In another embodiment, the reference electrode is a long-life reference electrode. In embodiments, the leakless reference electrode is a bipolar reference electrode.

In embodiments, the bipolar reference electrode described herein is miniature, e.g., less than about 100 μm, and leakless. In one embodiment, the reference electrode is less than about 2 mm, less than about 1 mm, or less than about 0.5 mm in diameter. In embodiments, the reference electrode fits in a cell in vivo. In embodiments, the reference electrode is less than about 100 μm, less than about 50 μm, less than about 25 μm, less than about 10 μm, or less than about 1 μm in diameter. In embodiments, the reference electrode is about 1 μm to about 100 μm, about 10 μm to about 100 μm, about 25 μm to about 100 μm, about 50 μm to about 100 μm, about 75 μm to about 100 μm, about 1 μm to about 75 μm, about 1 μm to about 50 μm, about 1 μm to about 25 μm, or about 1 μm to about 10 μm in diameter.

In another embodiment, the reference electrodes described herein maintain the high potential stability of the Ag/AgCl reference electrode. In embodiments, the reference electrode comprises a glass capillary tube, conductive wire sealed into the tip, and a silver/silver chloride wire in the other end of the tube. In embodiments the glass capillary tube is made of borosilicate or quartz. In embodiments, the conductive wire sealed into the tip is made of platinum.

A reference electrode for electrochemical measurements needs to hold the same well-defined potential over a long period of time in order for the electrochemical measurement to be valid. The first reference electrode was the standard hydrogen electrode (SHE), which has been arbitrarily assigned the value of 0.000 V. Since then, a number of different reference electrodes have been designed for use in different systems, such as the saturated calomel electrode (+0.241 V vs. SHE) and the silver/silver chloride (Ag/AgCl) reference electrode (+0.197 V vs. SHE). As it is significantly less harmful to the environment than the saturated calomel electrode, the Ag/AgCl reference electrode has become one of the most widely used reference electrodes for electrochemical measurements.

The Ag/AgCl reference electrode is a silver wire, anodized in a solution of Cl⁻ ions to produce AgCl on the surface of the wire. The wire is then encased in a hollow glass or plastic tube containing KCl of a high concentration (usually 1 M or higher) with a porous frit at the bottom that allows ions to pass through it. The connection to the electrochemical measurement device is made at the top of the bare silver wire, usually with a lead wire. Advantages of this reference electrode include a highly stable potential, being relatively non-toxic, and relatively simple and inexpensive to manufacture. However, due to the porous nature of the frit, there is a problem with silver ion leakage over extended use. While this reference electrode is labeled as non-toxic compared to other reference electrodes that have been common in the past, recent studies have shown that silver ions and nanoparticles can be toxic to cells and can interfere and affect redox reactions that are occurring on electrocatalysts. Additionally, it is difficult to manufacture a miniaturized version, due to the need for a frit.

Provided herein is a bipolar reference electrode which is similar in construction to the Ag/AgCl reference electrode; however, instead of a porous frit there is a piece of platinum wire. The bipolar reference electrode is constructed by sealing a piece of platinum wire into the bottom of the glass tubing. The tube is then filled with KCl (usually 1 M), and then an anodized silver/silver chloride wire is inserted into the top. Connection is made to the electrochemical system by attaching a piece of copper tape to the top, un-anodized portion of the silver wire. The use of the platinum wire means that there is no transfer of ions occurring between the inner filling solution of the reference electrode and the sample solution. Instead, in order to maintain charge balance, a reaction occurs on both sides of the platinum wire. As a result of this bipolar reaction, instead of transferring ions in and out of the reference electrode to maintain charge balance, electrons are transferred. As the inner filling solution of the bipolar reference electrode is 1 M (or higher) KCl, the reaction occurring on the inner surface of the platinum wire will be either the conversion of oxygen to water, or water to oxygen, depending on which direction the electrons are flowing for the particular reaction being studied by the electrochemical system. If the sample solution being tested is aqueous, then the opposing reaction (water to oxygen or oxygen to water) will occur on the outer surface of the platinum wire. If the sample solution is organic, the reaction being driven on the outer surface of the platinum wire will depend upon the identity of the organic solvent.

In embodiments, the reference electrode is made of conductive materials such as gold, palladium, platinum, or carbon.

The advantage of using a platinum wire instead of a porous glass frit is that there should be no ion leakage. Platinum is very inert, and it would take a very high current being applied to cause the reaction occurring on the surface of the platinum wire to release platinum ions instead of turning over a species in solution. Additionally, the most likely reaction for an aqueous sample (water to oxygen and oxygen to water) is well-known to be not particularly sensitive to temperature fluctuations, so the reference electrode itself should be fairly insensitive to temperature fluctuations. Platinum wire is also incredibly easy to miniaturize, unlike a porous frit.

### III. Methods

In one embodiment, a method of measuring a target substance concentration in a sample comprising: contacting the sample comprising the target substance with a biosensor which comprises an enzyme electrode comprising an oxidoreductase immobilized on the electrode and a reference electrode; measuring a time-dependent change of an open circuit potential between the enzyme electrode and the reference electrode; and calculating the concentration of the target substance based on the time-dependent change of the open circuit potential. In embodiments, the biosensor further comprises a counter electrode. In embodiments, no potential is applied before measuring the time-dependent change of the open circuit potential. In another embodiment, a potential is applied before measuring the time-dependent change of the open circuit potential. In embodiments, a potential is applied before measuring the time-dependent change of the open circuit potential and the biosensor further comprises a counter electrode. In embodiments, a counter electrode is used when a potential is applied before measuring the time-dependent change of the open circuit potential. In embodiments, the potential is measured across the high input impedance between the working electrode and the reference electrode.

The term “open circuit potential” refers to the potential established between the working electrode and the environment, with respect to a reference electrode.

The term “time dependent change” may refer to dOCP/dt or to dOCP/d√t.

In embodiments, provided herein is a method for continuous measurement. In embodiments, the continuous measurement comprises a plurality of individual measurements. In embodiments, the time for measuring each of the plurality of individual measurements is less than about 60 seconds. In embodiments, the time for measuring each of the plurality of individual measurements is less than about 50 seconds, less than about 40 seconds, less than about 30 seconds, less than about 20 seconds, less than about 15 seconds, less than about 10 seconds, less than about 5 seconds, less than about 1 second, less than about 0.9 seconds, less than about 0.8 seconds, less than about 0.7 seconds, less than about 0.6 seconds, less than about 0.5 seconds, less than about 0.4 seconds, less than about 0.3 seconds, less than about 0.2 seconds, and less than about 0.1 seconds. In embodiments, the time each of the plurality of individual measurements is from about 50 seconds to about 60 seconds, from about 40 to about 60 seconds, from about 30 seconds to about 60 seconds, from about 20 seconds to about 60 seconds, from about 10 seconds to about 60 seconds, from about 5 seconds to about 60 seconds, from about 1 second to about 60 seconds, from a about 1 second to about 50 seconds, from about 1 second to about 40 seconds, from about 1 second to about 30 seconds, from about 1 second to about 20 seconds, from about 1 second to about 10 seconds, and from about 1 second to about 5 seconds. In embodiments, the time each of the plurality of individual measurements is from about 0.1 seconds to about 1 second, from about 0.2 seconds to about 1 second, from about 0.3 seconds to about 1 second, from about 0.4 seconds to about 1 second, from about 0.5 seconds to about 1 second, from about 0.6 seconds to about 1 second, from about 0.7 seconds to about 1 second, from about 0.8 seconds to about 1 second, from about 0.9 seconds to about 1 second, from about 0.1 seconds to about 0.9 seconds, 0.1 seconds to about 0.8 seconds, 0.1 seconds to about 0.7 seconds, 0.1 seconds to about 0.6 seconds, 0.1 seconds to about 0.5 seconds, 0.1 seconds to about 0.4 seconds, 0.1 seconds to about 0.3 seconds, and 0.1 seconds to about 0.2 seconds.

In embodiments, the target substance is glucose and the oxidoreductase is glucose dehydrogenase or glucose oxidase. In embodiments, the target substance is lactate and the oxidoreductase is lactate oxidase. In embodiments, the target substance is D-serine and the oxidoreductase is D-amino acid oxidase.

In embodiments, the sample is from a subject. The term “subject” refers to a mammal (e.g., a human) in need of a triglyceride analysis. The subject may include dogs, cats, pigs, cows, sheep, goats, horses, rats, mice, non-human mammals, and humans. The term “subject” does not necessarily exclude an individual that is healthy in all respects and does not have or show signs of elevated or lowered target substance. In embodiments, the sample is a biological sample.

As used herein, the term “physiological conditions” refers to the range of conditions of temperature, pH, and tonicity (or osmolality) normally encountered within tissues in the body of a living human.

Compositions or methods “comprising” or “including” one or more recited elements may include other elements not specifically recited. For example, a composition that “comprises” or “includes” a protein may contain the protein alone or in combination with other ingredients.

Designation of a range of values includes all integers within or defining the range, and all subranges defined by integers within the range.

Unless otherwise apparent from the context, the term “about” encompasses values within a standard margin of error of measurement (e.g., SEM) of a stated value or variations ±0.5%, 1%, 5%, or 10% from a specified value.

The singular forms of the articles “a,” “an,” and “the” include plural references unless the context clearly dictates otherwise.

Statistically significant means p≤0.05.

The disclosed subject matter is further described in the following non-limiting Examples. It should be understood that these Examples, while indicating preferred embodiments of the invention, are given by way of illustration only.

### DISCUSSION AND EXAMPLES

D-Serine is a major co-agonist at the n-methyl-D-aspartate receptor (NMDA) receptor for glutamatergic excitatory transmission in mammalian brain. In recent years, the correlation between the d-serine and neurological diseases such as Alzheimer's disease, depression and schizophrenia has been attracting attention (Madeira et al., 2015; Guercio et al., 2018; MacKay et al., 2019), and D-serine monitoring with high-spatial resolution, especially in the inter-synaptic cleft, is required for a deeper understanding of brain function and the elucidation of neurological diseases. In addition, D-serine is also attracting attention as a future biomarker for neurological diseases. The D-serine concentrations in the prefrontal cortex of rats and in the cerebrospinal fluid (CSF) of humans are significantly increased in Alzheimer's disease, depression, and hydrocephalus (Pernot et al., 2008; Madeira et al., 2015).

D-serine biosensing has been reported based on enzyme sensors using flavin adenine dinucleotide (FAD)-dependent D-amino acid oxidase (DAAOx) (EC 1.4.3.3). DAAOx is the enzyme that specifically recognizes D-amino acids, including D-serine, and catalyzes their oxidation using oxygen as its electron acceptor (Pilone, 2000). The DAAOx derived from Rhodotorula gracilis (R. gracilis) has a higher D-serine recognition ability than other pig and human-derived DAAOx, which enables D-serine biosensing in extracellular regions of the brain containing other D-amino acids such as D-aspartate (Pernot et al., 2008). Using DAAOx, D-serine biosensing systems that measure the current flowing by oxidizing hydrogen peroxide generated by the enzymatic reaction using oxygen as an electron acceptor at a Pt electrode based on amperometry principle have been developed (Pernot et al., 2008; Pernot et al., 2012; Polcari et al., 2014; Polcari et al., 2017; Perry et al., 2018; Campos-Beltrán et al., 2018). These sensors have achieved a responsivity of 2 s and a very good lower limit of detection of 16 nM for D-serine in vitro (Zain et al., 2010). Since oxygen is used as an electron acceptor in this principle, the effect of dissolved oxygen concentration in the measurement environment is noticeable in in vivo use.

Regarding principles for electrochemical enzyme sensor, amperometry, voltammetry, and impedimetric are used for converting enzymatic reactions into electric signals. Although these principles are suitable for enzymatic biosensors, however, the obtained signal basically depends on the electrode surface area. Namely, the sensor signal generated by amperometric principle is directly proportional to the size of the sensor, i.e. the larger sensors provide greater signal. Specifically, the catalytic current depends on the surface area of the sensing electrode. The challenge to downsizing electrochemical sensors employing amperometry is that the current (signal proportional to electrode size) also decreases. Therefore, there is a technical problem in miniaturization into a nano-size (Clausmeyer et al., 2016).

Meanwhile, open circuit potential (OCP)-based measurement has been attracted attention, and applied for biosensing which combined with enzymes (Katz et al., 2001; Lee and Cui, 2012). In the OCP measurement, bias current flows only little or on the order of femto amperes during the measurement, which enables nondestructive analysis and is expected to reduce the consumption of analytes due to excessive redox cycling. In addition, it has recently been shown that the OCP changes associated with the enzymatic reaction follow the Nernst equation, and importantly, are independent of the effects of electrode size and mass transfer (Percival and Bard, 2017; Smith et al., 2019). The development of biosensors that take advantage of these useful properties promises to be a technique for tracking molecular dynamics in vivo, ultra-small regions and non-invasive intracellular measurement, which has been difficult to achieve by conventional electrochemical measurement principle. Therefore, an enzyme sensor for D-serine measurement based on the OCP principle could be a breakthrough for in vivo D-serine monitoring, which has been yet to be developed. Studies of direct electron transfer (DET)-type enzyme glucose sensors based on OCP principle have been reported (Lee et al., 2019). When the circuit is opened, minimal current (10-15 femto amperes or less) flows in the circuit; therefore, there is no appreciable current flow. Thus, the most ideal configuration of OCP based enzyme sensor is the employment of DET-type redox enzymes. However, DAAOx is not capable of DET, as its cofactor FAD is deeply buried in the protein molecule, and hard to transfer electron with electrode, without the association of the presence of artificial electron acceptor. Moreover, the nature of DAAOx is that the primary electron acceptor is oxygen, which competes with the electron transfer from FAD to external synthetic electron acceptors.

### Example 1: dOCP/Dt Measurement in Biosensors

A method for monitoring target analyte concentration by monitoring the time dependent change of the open circuit potential (OCP) between working electrode where redox enzyme is immobilized, and counter electrode, in the presence of target analyte was developed. In other words, measuring differentiation of potential by time (dOCP/dt) by monitoring the difference of potential between the working electrode, where redox-enzymes are immobilized, and counter electrode, in the presence of target analyte.

The monitoring of dOCP/dt is carried out after the sample was injected to the solution where sensor is immersed. In embodiments, dOCP/dt is measured after the potential application between working and counter electrode. For the experiments described below, a gold electrode was used as the working electrode, a platinum electrode was used as the counter electrode, and a Ag/AgCl electrode was used as the reference electrode for potential application to the working electrode.

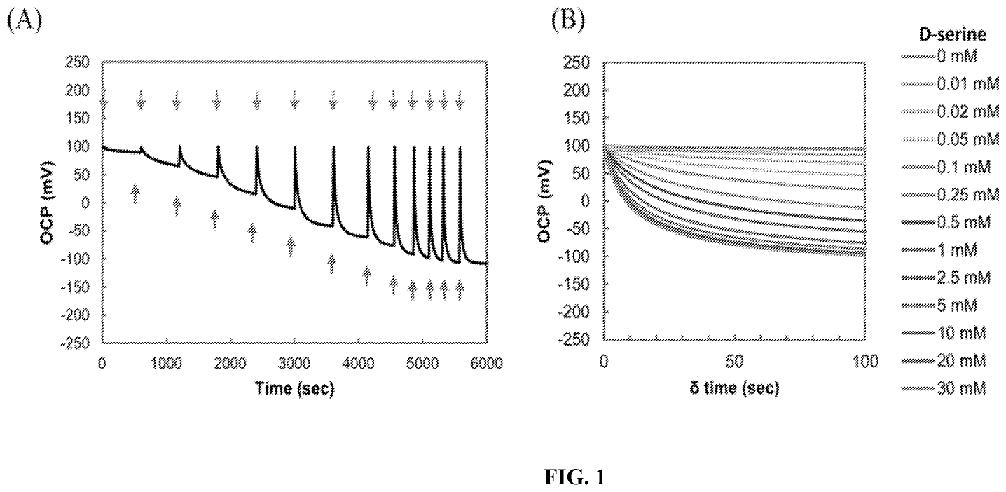

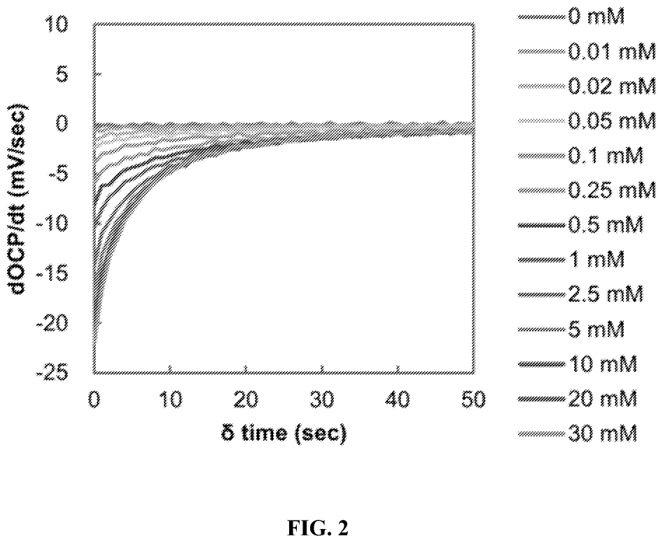

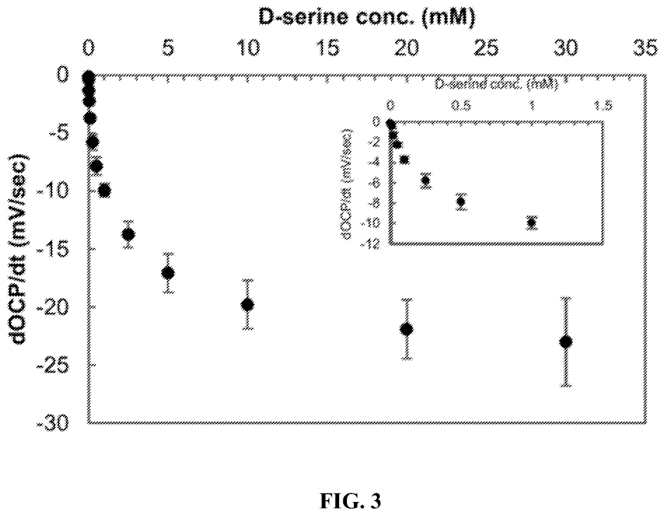

FIGS. 1-3 show the dOCP/dt based measurement of D-serine (D-Ser), using redox-mediator modified engineered D-amino acid oxidase (DAAOx). The redox mediator is 1-[3 (succinimidyloxycarbonyl) propoxy]-5-ethylphenazinium trifluoromethanesulfonate; amino-reactive phenazine ethosulfate (arPES). The engineered DAAOx is a mutant DAAOx with Gly52Val substitution, which repressed its oxidase activity by keeping catalytic activity for the oxidation of D-amino acid (reductive half reaction). The sensor is composed of a 3 mm diameter gold electrode as a working electrode, a platinum wire as a counter electrode, and a Ag/AgCl electrode as the reference electrode for potential application to working electrode. FIG. 1 shows that the OCP change is very slow and no steady state OCP was not observed at least within 100 sec, which is conventionally used as the value for OCP based sensing. Therefore, D-Ser sensing is not possible by monitoring OCP value changed upon the change of D-Ser concentration. FIG. 2 shows the time dependent change of dOCP/dt, and in any time, dOCP/dt value is dependent on the D-Ser concentration. However, FIG. 3 clearly demonstrates that by monitoring dOCP/dt values, D-Ser concentration can be measured. dOCP/dt values correlate to substrate concentration, not to a logarithmic concentration of substrate.

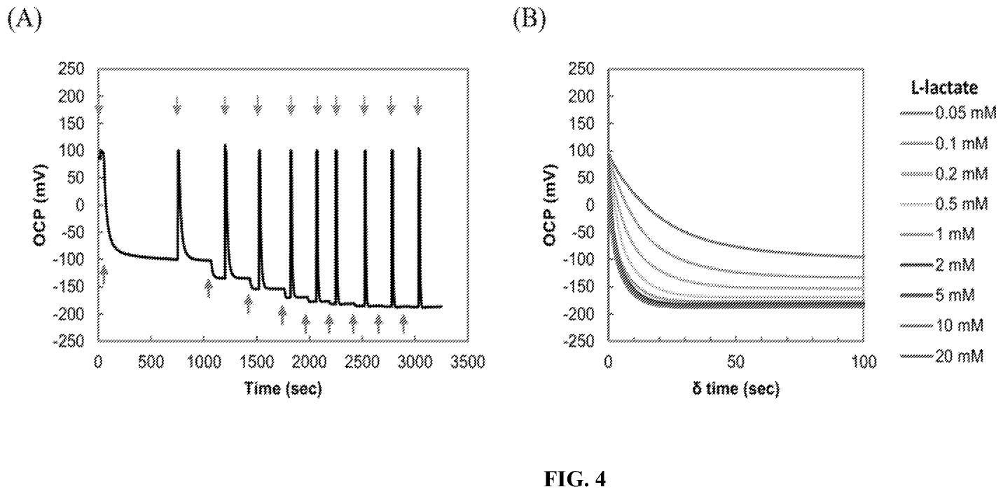

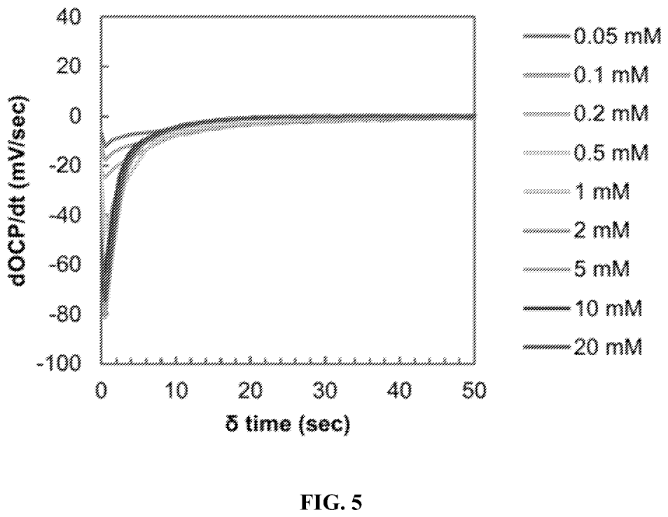

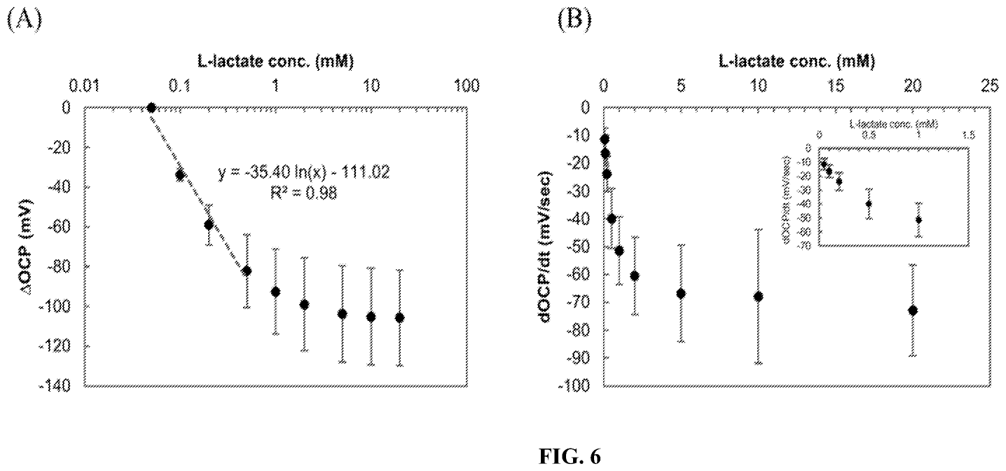

FIGS. 4-6 show the dOCP/dt based measurement of L-lactate, using PES modified engineered lactate oxidase (LOx). The engineered LOx is a mutant LOx with Ala96Leu and Asn212Lys substitutions, which repressed its oxidase activity by keeping catalytic activity for the oxidation of L-lactate (reductive half reaction), and additional Lys residue to be modified by PES. The sensor is composed of a 3 mm diameter gold electrode as a working electrode, a platinum wire as a counter electrode, and a Ag/AgCl electrode as the reference electrode for potential application to the working electrode. FIG. 4 shows that OCP change is very slow, and it took at least more than 20 seconds at a L-lactate concentration higher than 5 mM, and more than 50 sec at a L-lactate concentration lower than 20 mM. FIG. 5 shows the time dependent change of dOCP/dt, and in any time, dOCP/dt value is dependent on the L-lactate concentration. FIG. 6A shows the OCP value vs L-lactate concentration, which should be displayed in logarithmic of L-lactate concentration. FIG. 6B clearly demonstrates that by monitoring dOCP/dt values, L-lactate concentration can be measured. dOCP/dt values correlate to substrate concentration, not to a logarithmic concentration of substrate.

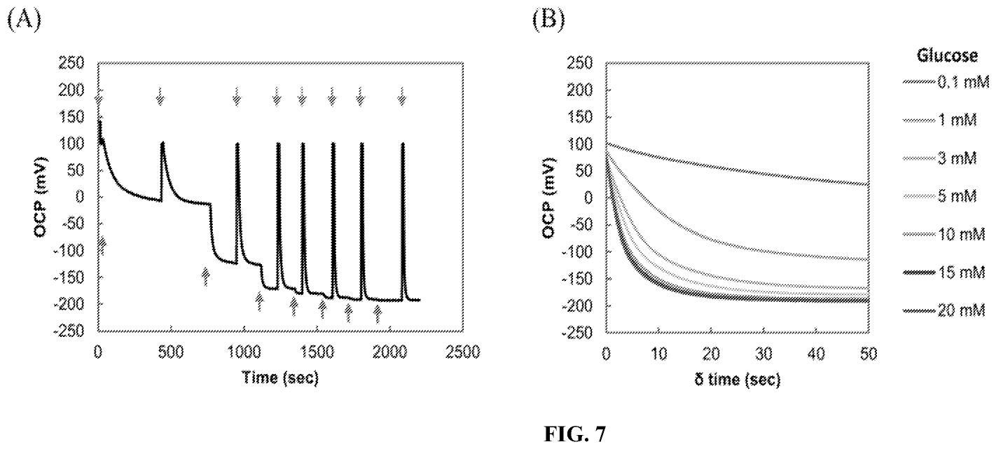

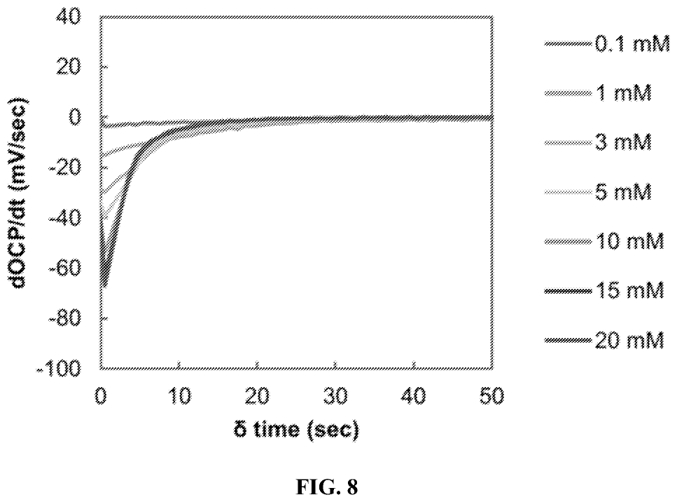

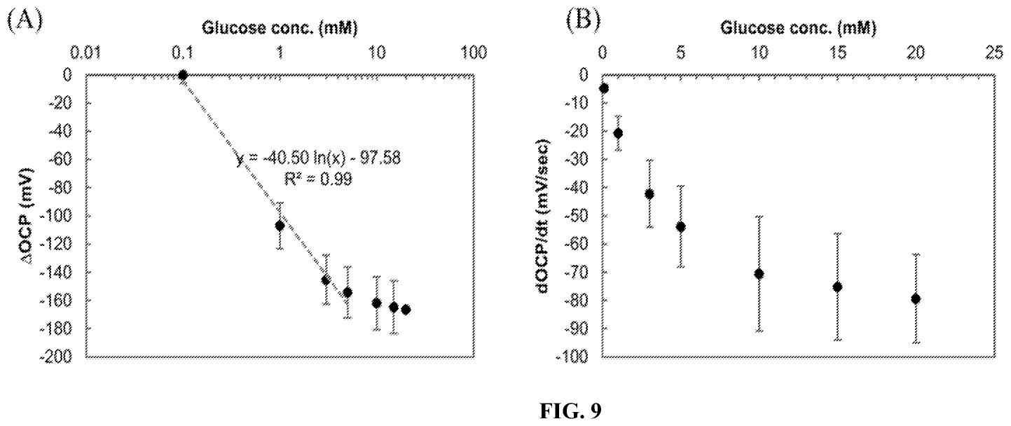

FIGS. 7-9 show the dOCP/dt based measurement of glucose, using PES modified fungi derived glucose dehydrogenase (GDH), which is inherently unable to transfer electron directly to electrode. The sensor is composed of a 3 mm diameter gold electrode as a working electrode, a platinum wire as a counter electrode, and a Ag/AgCl electrode as the reference electrode for potential application to working electrode. FIG. 7 shows that OCP change is very slow, and it took at least more than 20 seconds at a glucose concentration higher than 10 mM, and more than 50 sec at a glucose concentration lower than 5 mM. FIG. 8 shows the time dependent change of dOCP/dt, and in any time, dOCP/dt value is dependent on the glucose concentration. FIG. 9A shows the OCP value vs glucose concentration, which should be displayed in logarithmic of glucose concentration. FIG. 9B clearly demonstrates that by monitoring dOCP/dt values, glucose concentration can be measured. dOCP/dt values correlate to substrate concentration, not to a logarithmic concentration of substrate.

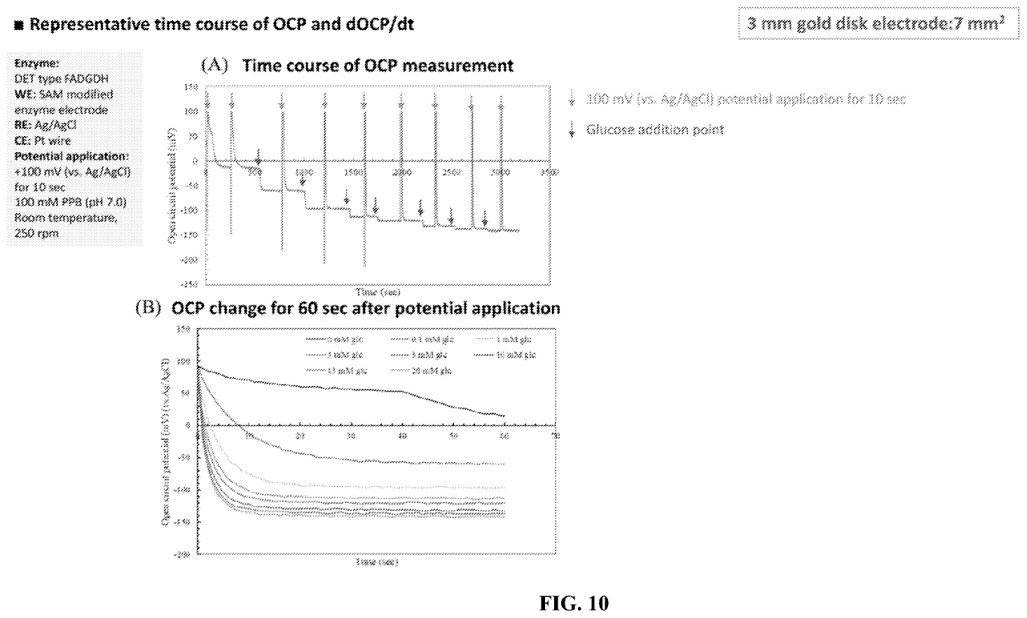

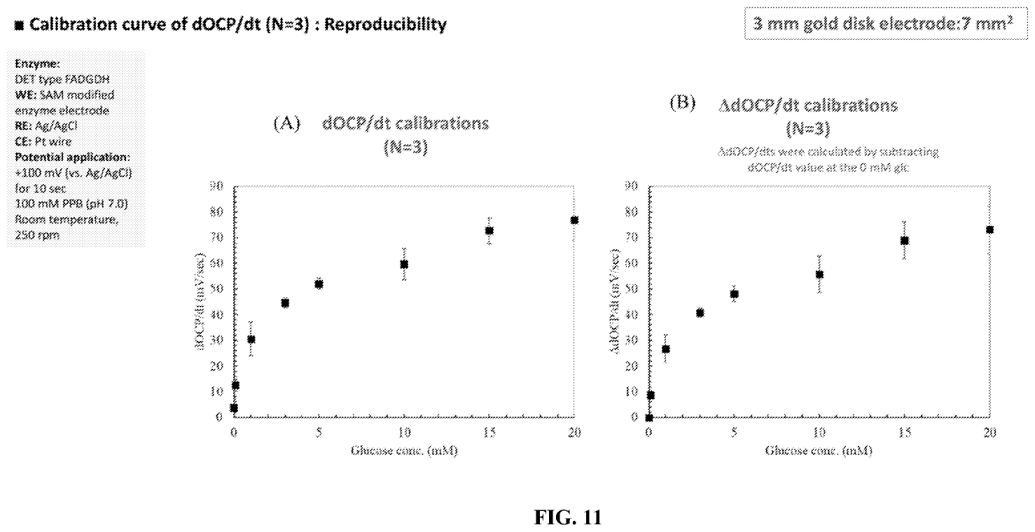

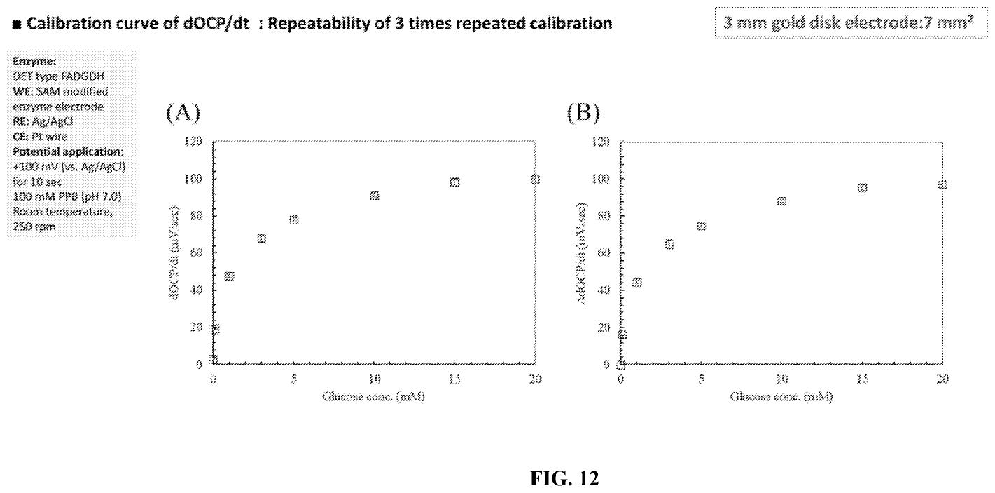

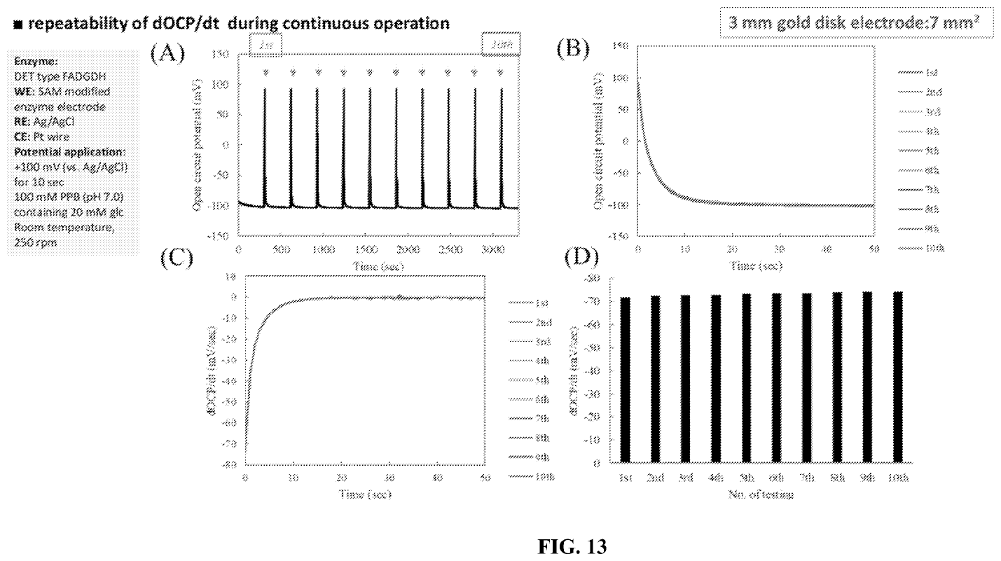

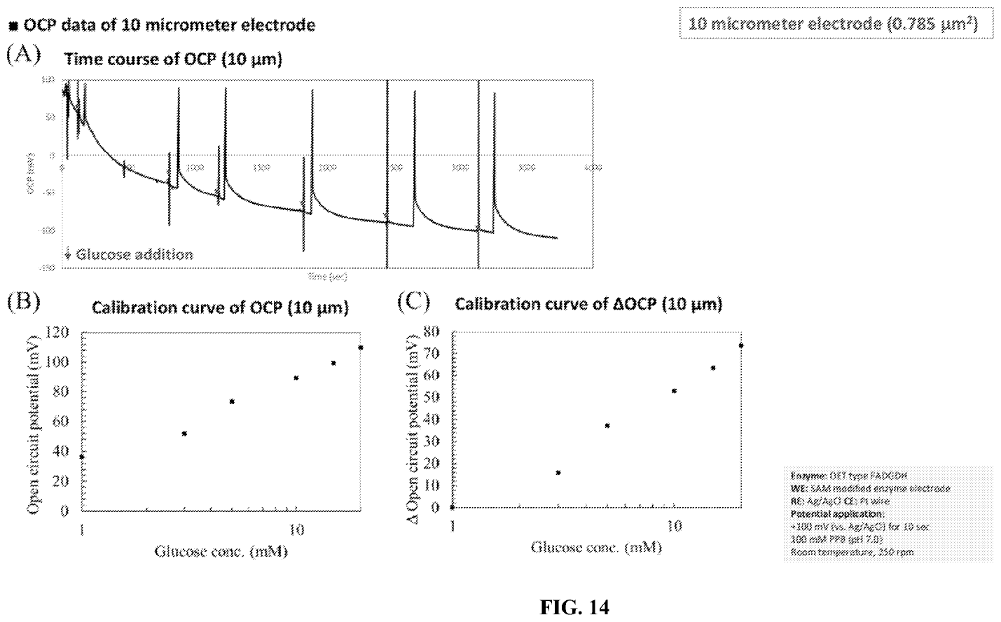

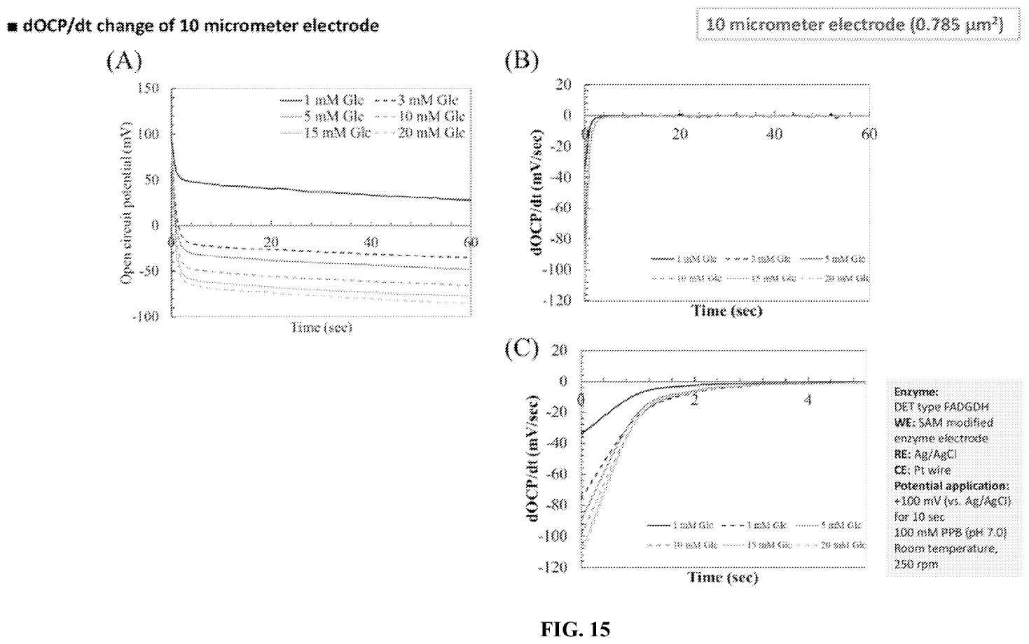

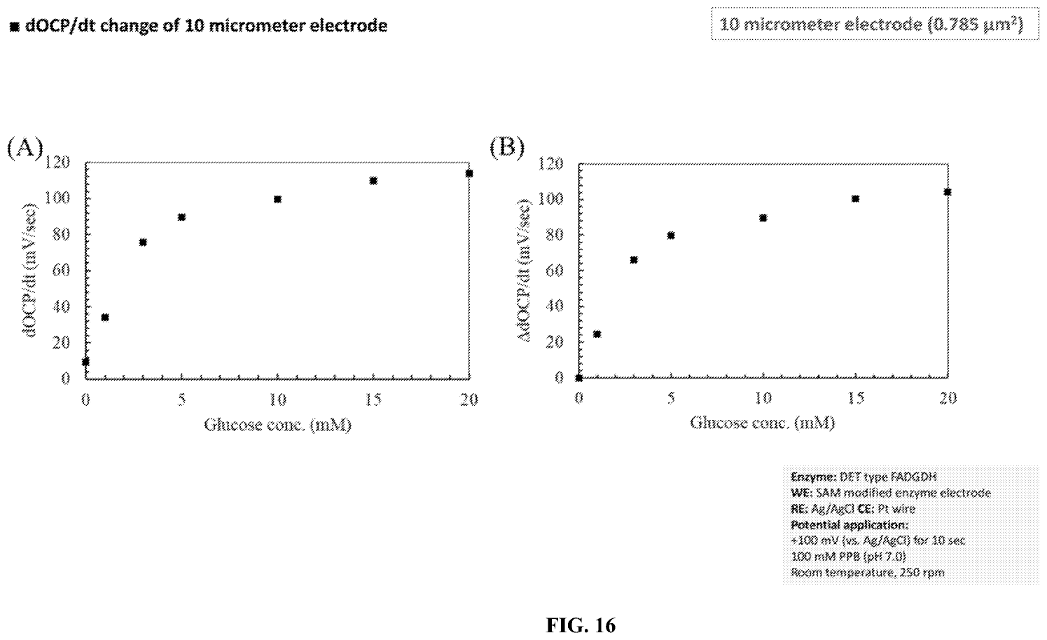

FIGS. 10-16 show the dOCP/dt based measurement of glucose, using direct electron transfer-type FAD-dependent glucose dehydrogenase (DET-FADGDH), which is able to transfer electrons directly to an electrode. The sensor is composed of a 3 mm diameter gold electrode (FIGS. 10-13) or 10 μm diameter gold electrode (FIGS. 14-16) as a working electrode, a platinum wire as a counter electrode, and a Ag/AgCl electrode as the reference electrode for potential application to working electrode. FIG. 10 shows that OCP change is slow, and it took at least more than 10 seconds at a glucose concentration higher than 5 mM, and more than 20 sec at a glucose concentration lower than 3 mM. FIGS. 11A and 11B show the calibration curves for glucose by monitoring dOCP/dt, or ΔdOCP/dt, respectively, which were monitored by 3 independent sensors with a 3 mm diameter gold electrode for working electrode, in order to show reproducibility. These results clearly demonstrates that by monitoring dOCP/dt values, glucose concentration can be measured. dOCP/dt values correlate to substrate concentration, not to a logarithmic concentration of substrate. FIGS. 12A and 12B show the calibration curves for glucose by monitoring dOCP/dt, or ΔdOCP/dt, respectively, which were monitored by 3 consecutive measurements with a 3 mm diameter gold electrode for working electrode, in order to show reproducibility. These results clearly demonstrates that by monitoring dOCP/dt values, glucose concentration can be measured. dOCP/dt values correlate to substrate concentration, not to a logarithmic concentration of substrate. FIG. 13 also shows the reproducibility of glucose monitoring using a 3 mm diameter gold electrode for working electrode. FIG. 14 shows that OCP change is slow when a 10 μm diameter gold working electrode was used, and no steady state OCP was observed within 1000 sec. If OCP values were chosen at a certain waiting time, OCP shows the linear relationship with logarithmic glucose concentration. However, FIG. 15 shows the time dependent change of dOCP/dt with 10 μm diameter gold working electrode, and in any time, dOCP/dt value is dependent on the glucose concentration. FIGS. 16A and 16B show the calibration curves for glucose by monitoring dOCP/dt, or ΔdOCP/dt, respectively, which were monitored by 3 consecutive measurements with a 10 μm diameter gold electrode as working electrode. These results clearly demonstrate that by monitoring dOCP/dt values, glucose concentration can be measured even with a 10 μm diameter gold electrode. dOCP/dt values correlate to substrate concentration, not to a logarithmic concentration of substrate.

These results clearly indicated that enzyme sensors monitoring dOCP/dt have significant advantages compared with amperometric sensors and previously reported OCP based sensors.

### Example 2: Bipolar Reference Electrodes

A reference electrode was developed to address two of the main issues with the traditional silver/silver chloride (Ag/AgCl) reference electrode: silver ion leakage and the difficulty of miniaturization. The use of a platinum wire sealed into the bottom of the reference electrode in place of a porous frit overcomes these issues, while maintaining the high potential stability of the Ag/AgCl reference electrode.

Testing of the reference electrode was performed using a gold macroelectrode as the working electrode. When a counter electrode was needed, a glassy carbon rod was used. Comparison of the performance of the bipolar electrodes described here and commercial Ag/AgCl reference electrodes was used to show that the bipolar electrodes perform just as well as the widely-used commercial Ag/AgCl reference electrodes do.

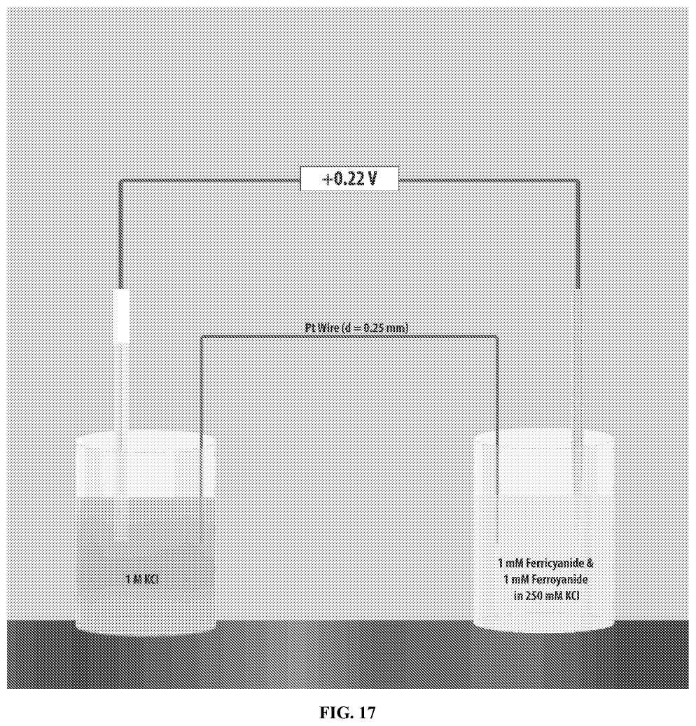

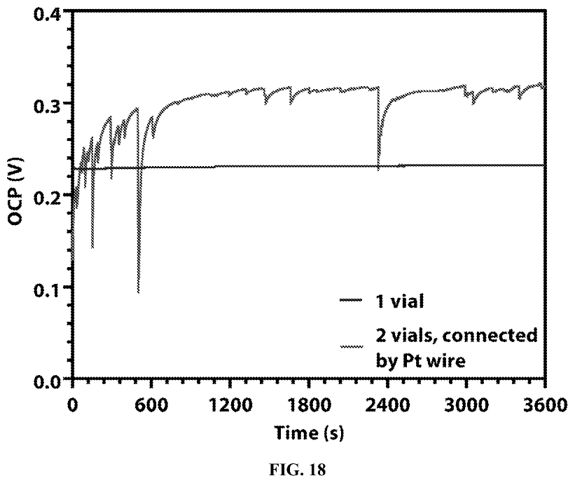

FIG. 17 is a schematic of an experiment performed to show that a platinum wire can be used as a bridge between 2 solutions to transfer electrons, instead of the usual method of using a frit or salt bridge to transfer ions. FIG. 18 shows data comparing the open circuit potential measured in this proof of concept experiment compared to a commercial Ag/AgCl reference electrode in the test solution, as the measurement would normally be taken. This experiment demonstrated that electrons can be used in the place of ions as the method for maintaining charge balance in the system.

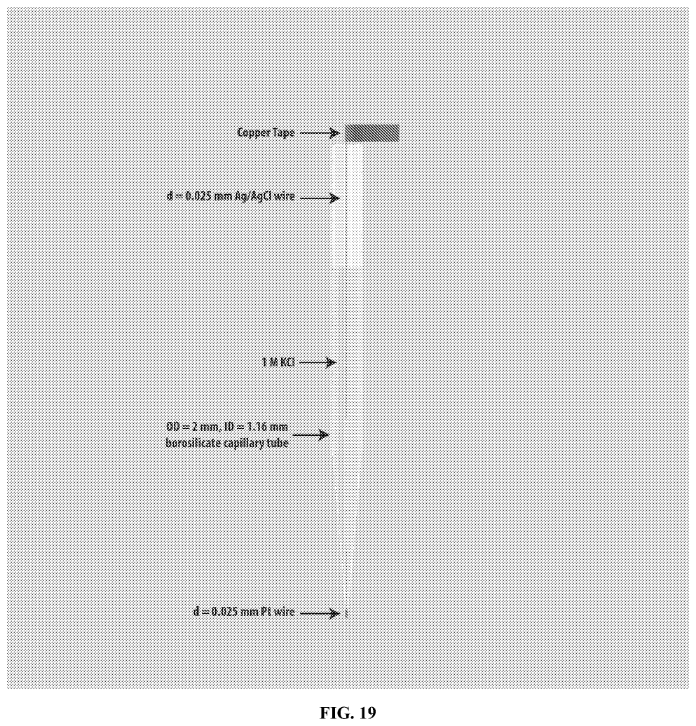

FIG. 19 is a schematic of the bipolar reference electrode. A conductive wire, such as platinum, is sealed into the tip of a glass capillary tube. The tube is then filled with an aqueous solution of KCl, and a silver/silver chloride wire is secured in the other end of the tube. Copper tape is used to make a connection with the electrochemical measurement system. In order to miniaturize this reference electrode, smaller capillary tubes and thinner wire is needed then shown in the schematic.

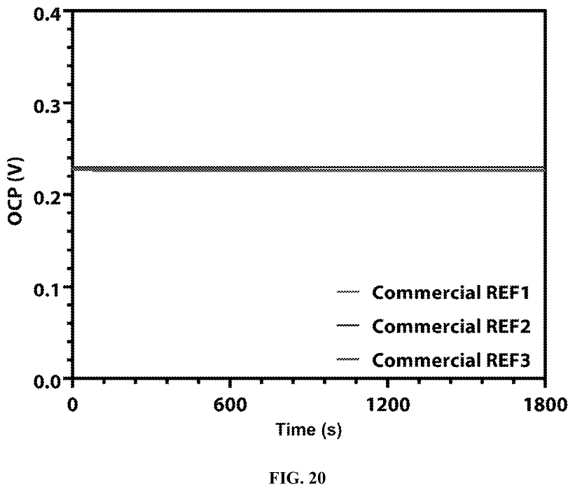

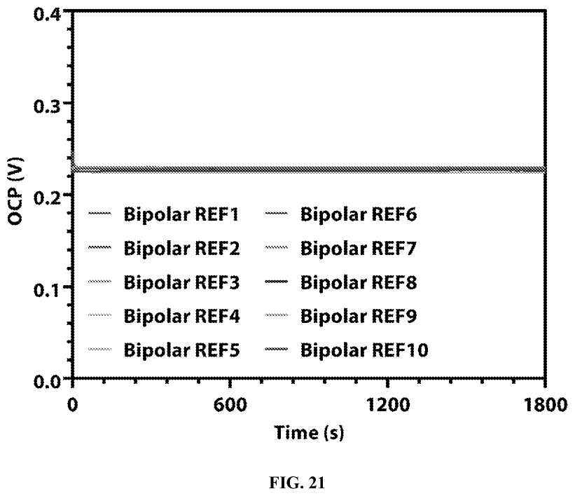

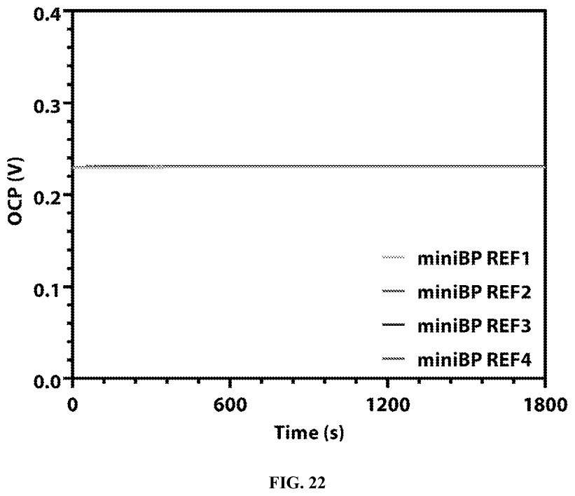

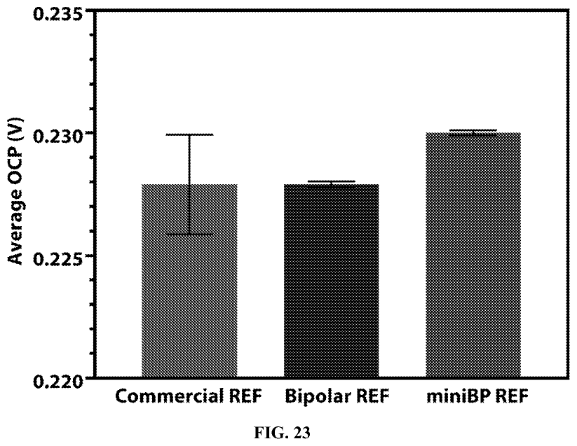

FIGS. 20-23 describe the results of open circuit potentiometry experiments performed in a solution of 1 mM ferricyanide and 1 mM ferrocyanide in 250 mM KCl using a gold macroelectrode for 30 minutes. In FIG. 20, the open circuit potentiometric trace is shown when three commercial Ag/AgCl reference electrodes are used. FIG. 21 shows the same when 10 bipolar reference electrodes (REFs) made according to the specifications of FIG. 19 are used. FIG. 22 shows the same when four miniature bipolar reference electrodes (miniBP REFs) are used. FIG. 23 shows a comparison of the average open circuit potential taken in each of these figures, showing that the bipolar reference electrodes measure comparable potentials to the commercial ones, varying by less than 2 mV from what the commercial reference electrodes measure, even when miniaturized to a platinum tip diameter of only 10 m.

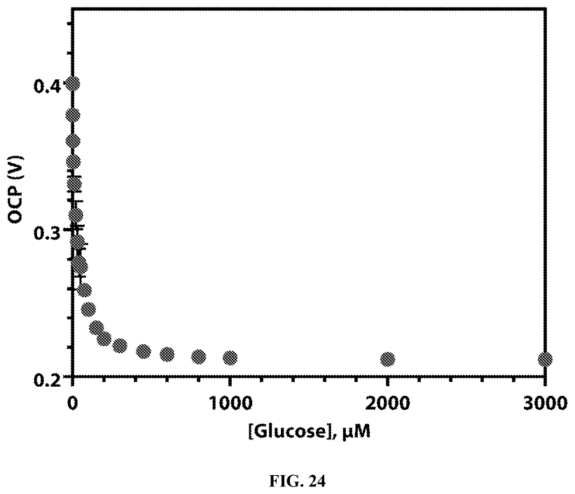

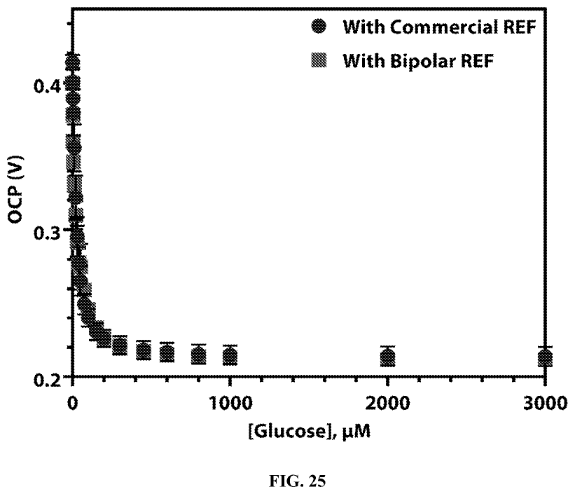

FIGS. 24 and 25 describe experiments performed in which the bipolar reference electrode was used in conjunction with a biosensor for detecting glucose using open circuit potentiometry. FIG. 24 shows the response of the glucose biosensor to increasing amounts of glucose, from 1 μM to 3 mM glucose. FIG. 25 shows a comparison of the response of the glucose biosensor to increasing amounts of glucose using the bipolar reference electrode and a commercial Ag/AgCl reference electrode. The results show that the bipolar reference electrode performs as well as the commercial reference electrode.

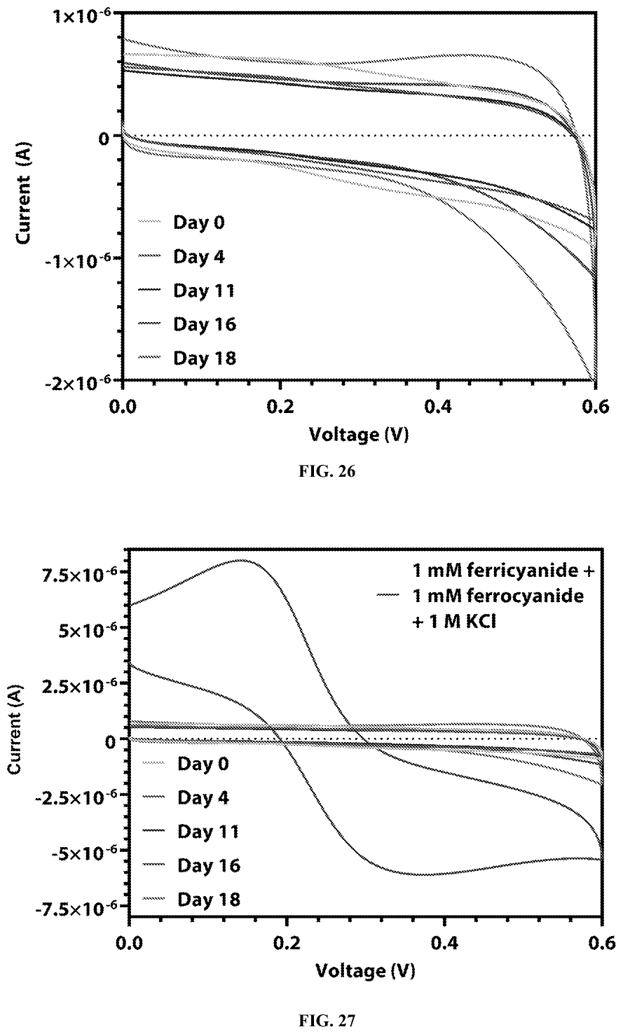

FIGS. 26 and 27 describe experiments performed to ensure that there is no leaking from the bipolar reference electrode. A bipolar reference electrode was filled with 1 mM ferricyanide and 1 mM ferrocyanide in 250 mM KCl and stored in a vial of 1 M KCl. Cyclic voltammograms were taken of the solution using a commercial Ag/AgCl reference electrode, a gold macroelectrode, and a glassy carbon counter electrode on day 0 (before the bipolar reference electrode was placed in solution) and periodically for the next 18 days. As shown in FIG. 27, the peaks on the cyclic voltammogram that would correspond with the leakage of ferri/ferrocyanide in to the 1 M KCl solution would appear around 0.16 V and 0.36 V. The lack of these two peaks indicates that none of the ferri/ferrocyanide leaked out of the tip of the bipolar reference electrode over 18 days.

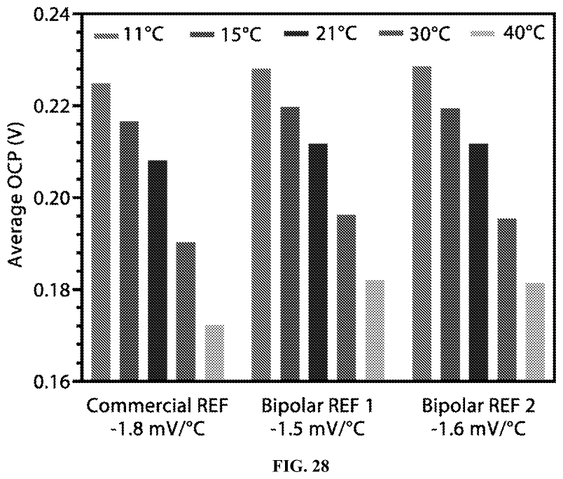

FIG. 28 describes the results of temperature studies. Open circuit potentiometry was performed for 30 minutes in a solution of 1 mM ferricyanide and 1 mM ferrocyanide in 250 mM KCl using a gold macroelectrode as the temperature of the solution was raised. One commercial Ag/AgCl reference electrode and two bipolar reference electrodes were tested in this manner. Comparison of the changes to the open circuit potential as temperature increased from 11° C. to 40° C. demonstrate that the bipolar reference electrodes perform as well as, if not a bit better than, the commercial Ag/AgCl reference electrode does in regards to maintaining a steady open circuit potential with changing solution temperature.

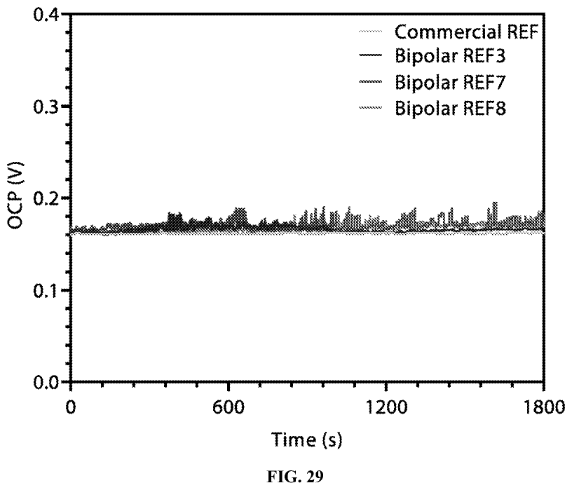

FIG. 29 is an experiment demonstrating that the bipolar reference electrode works in other sample solutions, namely solutions that are in organic solvent as well as those in aqueous solvent. The open circuit potential was measured for 30 minutes in a solution of 1 mM ferrocene in 100 mM tetrabutylammonium perchlorate in acetonitrile using a gold macroelectrode. Three commercial references and 3 bipolar reference electrodes were used, and the figure indicates that the bipolar reference electrode held a similar stable potential to the commercial Ag/AgCl reference electrodes even in organic solvent.

The results show that the bipolar reference electrode performs as well as the widely-used and acceptable silver/silver chloride reference electrode, and that it holds the advantages of ease of miniaturization and lack of ion leakage over the silver/silver chloride reference electrode.

### Example 3

In this study, a biosensing system for D-serine based on the DET-type OCP measurement principle was developed. To prepare DAAOx with a DET-type OCP measurement principle, DAAOx was converted into a quasi-DET-type enzyme by modification with redox mediator. AR. gracilis derived DAAOx mutant, Gly52Val (G52V), was employed, which was reported to maintain its reductive half-reaction with substrate, but is greatly reduced its oxidative half reaction with oxygen as an electron acceptor (Saam et al., 2010), although the availability of alternative electron acceptors has been yet to be investigated. This enzyme was immobilized on an electrode via self-assembled monolayer, and modified by an artificial electron acceptor (mediator), amine-reactive phenazine ethosulfate (arPES). A quasi-DET-type DAAOx immobilized electrode was constructed, and electrochemical monitoring of D-serine based on the OCP measurement principle was attempted, following a previous report (Hatada et al., 2018). Among several methods to analyze D-serine dependent OCP change, a transient potentiometric method was attempted, which provided fast and sensitive continuous D-serine monitoring using a quasi-DET-type DAAOx immobilized electrode.

D-serine, D-aspartate, L-serine and L-glutamate were purchased from Sigma-Aldrich (St. Louis, MO, USA). 4-aminoantipyrine (4-AA), KH₂PO₄, K₂HPO₄, (NH₄)₂SO₄, Na₂HPO₄, MgSO₄, NaCl, and ethanol were obtained from Kanto Kagaku (Tokyo, Japan). Imidazole, glycerol, lactose monohydrate, and kanamycin sulfate were purchased from FUJIFILM Wako Pure Chemical (Osaka, Japan). LB broth and ultrafiltration filter (Amicon Ultra-15 Centrifugal Filter Unit 30-K) were purchased from Merck Millipore (Billerica, MA, USA). 2,6-Dichloroindophenol (DCIP), Phenazine methosulfate (PMS), N-Ethyl-N-(2-hydroxy-3-sulfopropyl)-3-methylaniline sodium salt (TOOS), 3,3′-Dithiobis[N-(5-amino-5-carboxypentyl) propionamide-N′,N′-diacetic acid] dihydrochloride (Dithiobis (C₂-NTA)) and 1-[3 (succinimidyloxycarbonyl) propoxy]-5-ethylphenazinium trifluoromethanesulfonate (amine-reactive phenazine ethosulfate) were purchased from Dojindo Laboratories (Kumamoto, Japan). The restriction enzymes XbaI, HindIII and DpnI were purchased from New England Biolabs (MA, USA). PrimeSTAR Max DNA Polymerase and DNA ligation kit was purchased from TaKaRa Bio (Kyoto, Japan). KOD plus NEO DNA polymerase was purchased from TOYOBO (Osaka, Japan). The FastGene Gel/PCR Extraction Kit was purchased from Nippon Genetics (Tokyo, Japan). Peroxidase was purchased from Amano Enzyme (Gifu, Japan). All chemicals were of reagent grade. 1-mL HisTrap HP was purchased from Cytiva/Global Life Sciences Solutions (MA, USA). Gold (Au) disk electrodes (φ3 mm, surface area: 7 mm²), platinum (Pt) wire, and a silver/silver chloride (Ag/AgCl) reference electrode were purchased from BAS Inc. (Tokyo, Japan). Potentiostats, SP-50, SP-150, and VSP from Bio-Logic (Claix, France) were used for the electrochemical experiments.

Construction of Expression Vector for DAAOxs

According to Sonia et al., 2001, a gene of N-terminal His-tagged DAAOx wild-type (WT) from R. gracilis including T7 promoter and ribosome binding site was synthesized (Integrated DNA Technologies). The synthesized gene was digested by XbaI and HindIII, subsequently purified using the FastGene Gel/PCR Extraction Kit, and inserted into the XbaI-HindIII site of the pET30c vector to obtain an expression vector for N-terminal His-tagged DAAOx WT (DAAOx WT). An expression vector for DAAOx G52V (Saam et al., 2010; Rosini et al., 2011) was constructed by site-directed mutagenesis by quick-change PCR. PCR was performed using the DAAOx G52V forward primer (5′-GAC TTT CGC TTC ACC ATG GGC TGT CGC GAA TTG G-3′) (SEQ ID NO: 1) and DAAOx G52V reverse primer (5′-GAA AGG CGT CCA ATT CGC GAC AGC CCA TG-3′) (SEQ ID NO: 2) with template of the expression vector for DAAOx WT. The PCR product was purified by the FastGene Gel/PCR Extraction Kit, and digested by DpnI to remove the templated plasmid. The digested sample was used for transformation of Escherichia coli (E. coli) DH5α, and plasmid was extracted by cultured transformant. Correct insertion of the DAAOx WT gene into the XbaI-HindIII site on the pET30c vector, along with mutation to Gly52Val, were both confirmed by sequence analysis (Integrated DNA Technologies).

Recombinant Production of D-Amino Acid Oxidases

E. coli low background strain (LOBSTR) (Andersen et al., 2013) from Kerafast Inc. (MA, USA) was transformed with expression vectors for DAAOx. Transformed E. coli LOBSTR was grown aerobically for 12 h in 3 mL of Luria-Bertani (LB) medium containing 50 μg/mL kanamycin at 37° C. as a pre-cultivation. For the main cultivation, 1 mL of precultures were inoculated into 100 mL ZYP-5052 medium (0.5% glycerol, 0.05% glucose, 0.2% lactose, 50 mM KH₂PO₄, 25 mM (NH₄)₂SO₄, 50 mM Na₂HPO₄, and 1 mM MgSO₄) containing 50 μg/mL kanamycin in 500-mL baffled flask and cultured aerobically for 24 h at 30° C. under autoinduction system. Cells were harvested by centrifugation (10,000×g, 4° C., 20 min) and disrupted by a French pressure cell press in 20 mM potassium phosphate buffer (PPB) (pH 8.0) including 500 mM NaCl. After centrifugation (10,000×g, 4° C., 10 min) and ultracentrifugation (130,000×g, 4° C., 60 min), the supernatant was used as the soluble fraction and subjected to the Ni²⁺ affinity column chromatography using ÄKTA pure system and a 1-mL HisTrap HP column (both from Cytiva/Global Life Sciences Solutions, MA, USA) with a flow rate of 1 mL/min and a pre-column pressure of less than 0.5 MPa. The soluble fraction was injected into a 1-mL HisTrap HP column, which was equalized with 20 mM PPB (pH 8.0) containing 500 mM NaCl, and washed with 20 column volumes of 20 mM PPB (pH 8.0) containing 500 mM NaCl. Thereafter, DAAOxs were eluted by increasing the concentration of imidazole in the elution buffer to 500 mM over 30 column volumes with monitoring absorbance at 280 nm and 450 nm. In this step, each 1 mL elution was collected and evaluated by SDS-PAGE. Eluted fractions expected to contain DAAOx were concentrated and buffer-exchanged with Amicon 30-K, and stored at −80° C. until use.

Enzyme Activity Assay

An oxidase activity assay was conducted in 100 mM PPB (pH 8.0) containing 1.5 mM 4-AA, 1.5 mM TOOS, 2.0 U/mL peroxidase as final concentrations, and various concentrations of substrate. The formation of quinoneimine-dye caused from hydrogen peroxide production was measured by monitoring at 555 nm based on the molar absorption coefficient of TOOS (39.2 mM⁻¹cm⁻¹) for determination of oxidase activity of DAAOxs. Dye-mediated dehydrogenase activity was measured using PMS as a primary electron acceptor and DCIP as a secondary electron acceptor and as a color indicator. In detail, enzyme was mixed with 6 mM PMS, 0.06 mM DCIP and various concentration of D-serine in 100 mM PPB (pH 8.0) and absorbance change at 600 nm which associated with DCIP (the molar absorption coefficient: 16.3 mM⁻¹cm⁻¹) reduction. One unit of enzyme specific activity was defined as the amount of enzyme necessary to catalyze 1 μmol of substrate per minute. These assays were performed in triplicate at 37° C. Protein concentrations were determined by Bradford assay (Bio-Rad Laboratories, CA, USA).

Electrode Fabrications

Au disk electrodes (φ3 mm, surface area: 7 mm²) were polished and washed with ethanol before incubating them in 50 μM C₂-NTA, which was dissolved in ethanol overnight at 25° C. to form a self-assembled monolayer (SAM) of NTA. The NTA-SAM electrodes were then washed with distilled water and incubated in a solution of 40 mM NiCl₂ for 2 h. The prepared electrodes were then washed with distilled water and enzymes were immobilized by incubation in 1 mg/mL DAAOx WT or G52V for overnight at 4° C. Enzyme-immobilized electrodes were incubated in 1.67 mM arPES dissolved in 20 mM tricine buffer (pH 8.0) at room temperature for 30 min for on-site arPES modification. After on-site modification, the electrodes were washed with 20 mM PPB (pH 8.0) to remove any unmodified arPES. All electrodes were stored at 4° C. until further use.

Electrochemical Evaluation

The constructed electrodes were evaluated using a 10-mL electrochemical-measurement cell, with the Ag/AgCl as the reference electrode and Pt wire as counter electrode, respectively. The 100 mM PPB (pH 8.0) in the electrochemical-measurement cell was always agitated at 250 rpm by a magnetic stirrer. The potentiostat SP-150 or VSP (Bio-Logic, Claix, France) was used for electrochemical evaluation. On-site arPES modification was characterized by cyclic voltammetry (CV) with scan rate at 100 mV/s using constructed enzyme electrodes before and after modification procedure. An amperometric measurement was carried out by applying potential 0 mV (vs. Ag/AgCl) and monitoring the current. A batch-wise OCP measurement was carried out by applying potential at 100 mV vs. Ag/AgCl to enzyme electrode for 0.1 s, and subsequently monitoring the OCP change at enzyme electrodes. For continuous OCP operation, cycle of potential application (100 mV vs. Ag/AgCl for 0.1 s) and OCP monitoring (1.9 s) was alternated. In all electrochemical evaluation, the reproducibility was tested by using independently constructed three enzyme electrodes.

Results

Recombinant production and characterization of DAAOxs

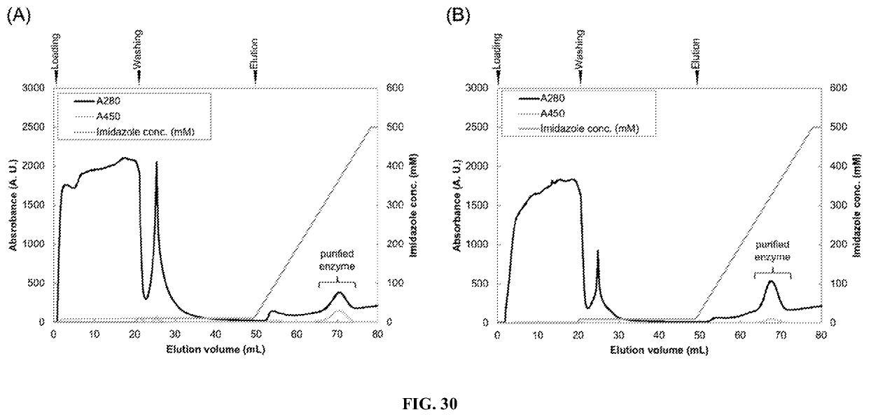

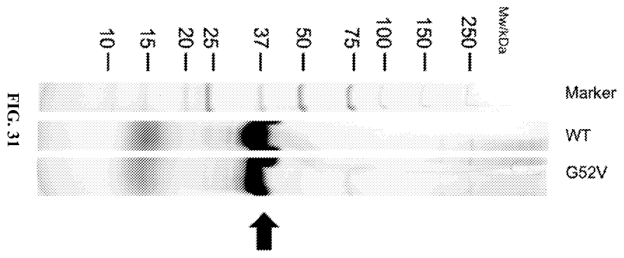

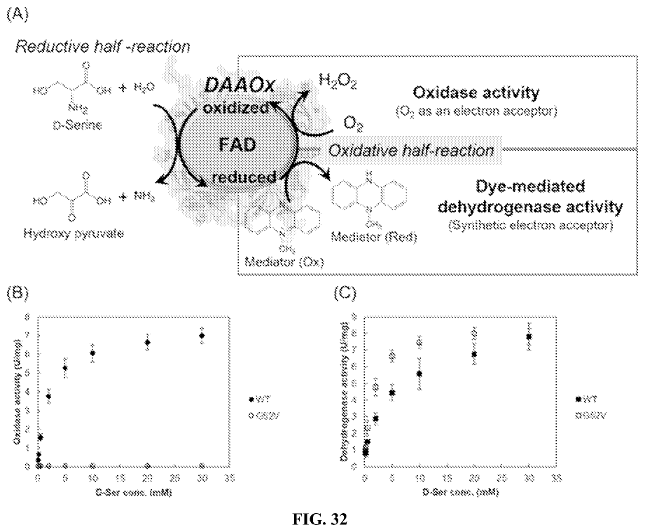

The E. coli LOBSTR strain transformed with the constructed vector was cultured, and the cellular soluble fraction was subjected to Ni²⁺ affinity column chromatography, which showed significant increasing of absorbance at both of 280 nm and 450 nm which indicating FAD-containing protein elution, in the fraction containing more than 200 mM imidazole (FIG. 30). The purified proteins showed a single band near the theoretical molecular weight of DAAOxs as 41.8 kDa in SDS-PAGE analysis (FIG. 31). This indicated that the DAAOxs were successfully purified. The DAAOx G52V mutant has been reported to greatly reduce its oxidative half reaction using oxygen as an electron acceptor, by keeping its reductive half reaction, which was confirmed by the formation of reduced FAD in the presence of substrate (Saam et al., 2010; Rosini et al., 2011). However, the availability of synthetic electron acceptors or mediators for oxidative half reaction in this mutant enzyme has not yet been investigated, which is necessary to know for further bioelectrochemical applications. Therefore, the dye-mediated dehydrogenase activity of DAAOx G52V mutant was investigated using D-serine as a substrate (FIG. 32A). As was reported, DAAOx G52V showed negligible oxidase activity to D-serine (FIG. 32B), whereas the wild type DAAOx showed an obvious oxidase activity in the presence of D-serine as the substrate, which was confirmed by the formation of hydrogen peroxide. By analyzing these substrate dependent enzymatic activities using Eadie-Hofstee plot (Table 1), Vₘₐₓ and Kₘ of DAAOx WT were calculated as 7.3±0.5 U/mg and 1.9±0.2 mM. Although the oxidase activity of DAAOx G52V was observed in subtle levels, only the maximum activity value was given (0.06±0.001 U/mg at 2 mM D-serine), because it did not follow a Michaelis-Menten-type saturation curve with respect to D-serine concentration. In contrast, the dye-mediated dehydrogenase activity of DAAOx G52V mutant was comparable to WT (FIG. 32C); Vₘₐₓ of DAAOx WT and G52V were 6.5±1.0 U/mg and 8.4±0.4 U/mg, and Kₘ of DAAOx WT and G52V were 1.8±0.3 mM and 1.4±0.4 mM, respectively (Table 1). From these measurements, it was confirmed that the DAAOx G52V mutant is a unique enzyme, virtually “dehydrogenase,” which almost lost its oxidase activity, whereas maintained its dye-mediated dehydrogenase activity using artificial synthetic electron acceptors (mediators), which was suitable for further bioelectrochemical application.

TABLE 1
Enzymatic properties of D-amino acid oxidase (DAAOx)
wild-type (WT) and Gly52Val (G52V) mutants.

| | | | Dye-mediated | |
|---|---|---|---|---|
| | Oxidase activity | | dehydrogenase activity | |
| | Vₘₐₓ (U/mg) | Kₘ (mM) | Vₘₐₓ (U/mg) | Kₘ (mM) |
| WT | 7.3 ± 0.5 | 1.9 ± 0.2 | 6.5 ± 1.0 | 1.8 ± 0.3 |
| G52V | 0.06 ± 0.001* | n.d. | 8.4 ± 0.4 | 1.4 ± 0.4 |

In Table 1, an oxidase activity was measured by using a dye-coloration reaction associated with hydrogen peroxide produced. A dye-mediated dehydrogenase activity was measured using phenazine methosulfate and 2,6-Dichloroindophenol (DCIP) with the fading of DCIP as an indicator. In both activity measurements, 1 U was defined as the amount of enzyme that catalyzed 1 μmol of substrate per minute. Details of the measurements are described in the section 2.4 in Materials and Method. A maximum enzyme activity (Vₘₐₓ) and a Michaelis constant (Kₘ) were calculated from the obtained D-serine concentration-dependent activity changes by Eadie-Hofstee plot. All measurements were performed in triplicate (n=3), and the average and standard deviations were shown in table. The n.d. in table means not determined.

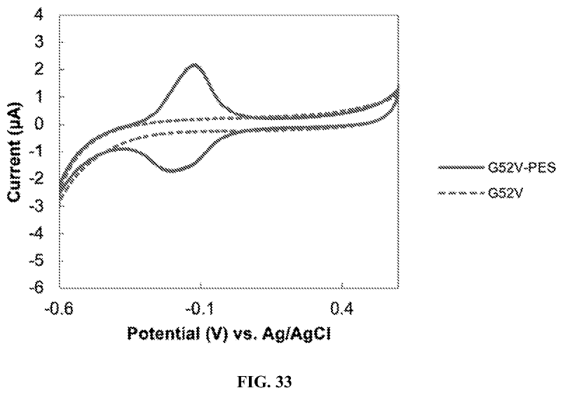

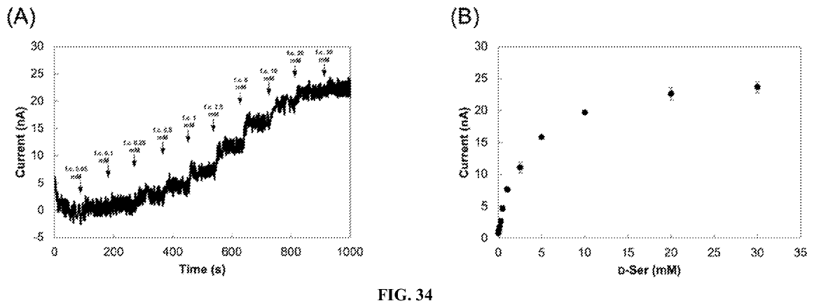

On-site modification of the DAAOx with arPES after immobilization on the Au electrode was performed as described previously (Takamatsu et al., 2021). The arPES modification was evaluated by performing CV of DAAOx G52V-immobilized electrodes, before and after arPES modification. Clear redox peaks were observed after the arPES modification (FIG. 33), and the redox potential was consistent with previously reported values (Hatada et al., 2018). These results suggested that PES molecules were successfully modified on the enzyme immobilized on the electrode. The chronoamperometry measurement was performed with PES-modified DAAOx G52V immobilized electrode, in the absence of addition of mediator in the solution applying OV vs Ag/AgCl, showing D-serine concentration dependent current increase (FIG. 34). A maximum steady current (Iₘₐₓ) and an apparent affinity (Kₘ₍ₐₚₚ₎) against D-serine were calculated as 24.3±0.8 nA and 2.2±0.3 mM, respectively, by Eadie-Hofstee plot. These results suggested that PES was modified with lysine residues that accept electrons from FAD, and that DAAOx G52V became the quasi-DET-type enzyme.

Quasi-DET-Type OCP-Based D-Serine Measurement

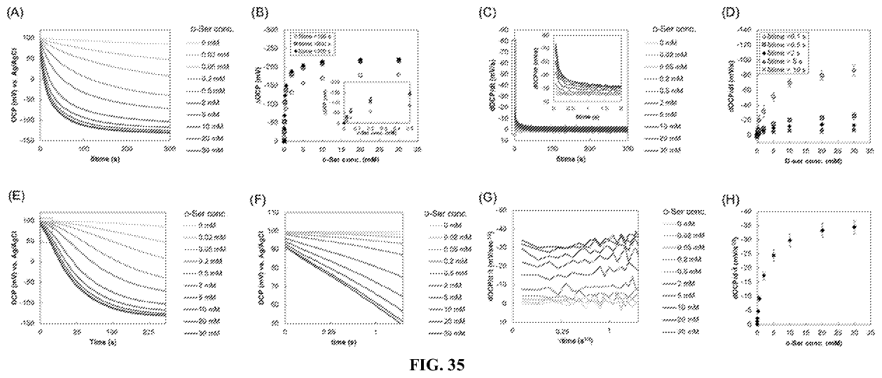

The PES-modified DAAOx G52V-immobilized electrode was used for D-serine measurement based on the quasi-DET-type OCP principle. In this experiment, the discharge (initialization of electrode potential) is carried out by applying 100 mV vs. Ag/AgCl over the first 0.1 s, and the OCP for the following 300 s was measured every 0.1 s. The results showed that the OCP maintained almost the same value as the initialized potential (100 mV vs. Ag/AgCl) in the absence of the substrate D-serine. In the presence of D-serine, the OCP decreased significantly with D-serine concentration-dependent manner (f.c. 0.02, 0.05, 0.2, 0.5, 1, 2, 5, 10, 20, 30 mM) (FIG. 35A). However, it took several minutes to reach a steady-state OCP value, especially for D-serine concentrations lower than 5 mM. Consequently, the calibration curve for D-serine concentration showed a sampling time dependence, especially at D-serine concentration lower than 0.5 mM (FIG. 35B and Table 2). Meanwhile, the slope of time course of OCP decrease obviously showed D-serine concentration dependency. Therefore, the kinetics of OCP change were examined and the transient of OCP (dOCP/dt) (FIG. 35C, D) and dOCP/d√t (FIG. 35F, G) were calculated.

TABLE 2
D-Serine response characteristics of amine-responsive
phenazine ethosulfate (PES)-modified D-amino acid
oxidase (DAAOx) Gly52Val (G52V) mutant
immobilized electrode based on the
steady-state open circuit potential (OCP).

| Sampling time | ΔOCPₘₐₓ (mV) | Kₘ₍ₐₚₚ₎ (mM) |
|---|---|---|
| 100 s | −173.0 ± 9.4 | 0.43 ± 0.04 |
| 200 s | −204.1 ± 8.5 | 0.15 ± 0.01 |
| 300 s | −205.5 ± 7.9 | 0.09 ± 0.01 |

From the results of FIG. 35B, a maximum ΔOCP (ΔOCPₘₐₓ) and the apparent affinity for D-serine (Kₘ₍ₐₚₚ₎) were calculated using the Eadie-Hofstee plot. Each value was shown as the average and standard deviation of the triplicate measurements (n=3).

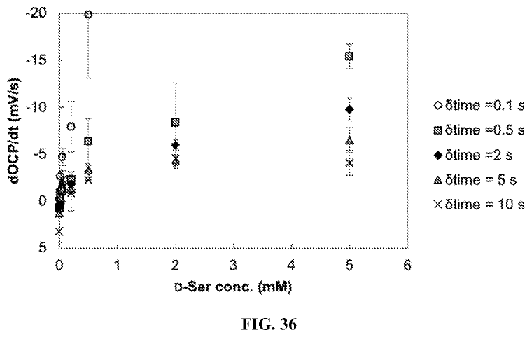

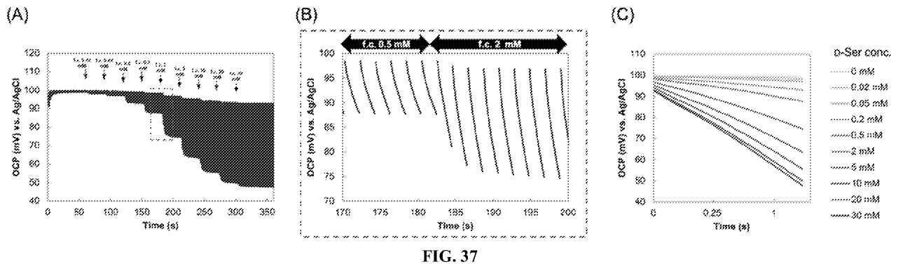

In the analysis of the kinetic OCP change, dOCP/dt showed a sharp change in the initial 2 seconds of the measurement, and dOCP/dt values showed D-serine concentration dependency (FIG. 35E). Although, dOCP/dt varied in a time-dependent manner (FIGS. 35C, D, and E and Table 3) within 2 seconds, after 5 seconds, steady state values were observed, and the calibration curve for D-serine concentration showed no sampling time dependence, at the concentration range from 0.5-5 mM, with LOD=0.5 mM (FIG. 36). These results indicate that the dOCP/dt based D-serine monitoring, will be suitable for measurement with time resolution longer than 5 seconds and D-serine concentration lower than 1 mM.

TABLE 3
D-serine response characteristics of
amine-responsive phenazine ethosulfate
(PES)-modified d-amino acid oxidase (DAAOx)
Gly52Val (G52V) mutant immobilizedelectrode based
on the open circuit potential change rate (dOCP/dt).

| Sampling time | dOCP/dtₘₐₓ (mV/s) | Kₘ₍ₐₚₚ₎ (mM) |
|---|---|---|
| 0.1 s | −100.0 ± 16.7 | 4.8 ± 2.9 |
| 0.5 s | −28.9 ± 5.8 | 4.5 ± 3.1 |
| 2 s | −15.1 ± 0.6 | 2.6 ± 0.6 |
| 5 s | −8.5 ± 0.6 | 1.1 ± 0.9 |
| 10 s | −5.9 ± 0.4 | 1.4 ± 0.7 |

From the results of FIG. 35D, a maximum dOCP/dt (dOCP/dtₘₐₓ) and the apparent affinity for D-serine (Kₘ₍ₐₚₚ₎) were calculated using the Eadie-Hofstee plot. Each value was shown as the average and standard deviation of the triplicate measurements (n=3).

The rate of change of OCP with respect to the square root of time, dOCP/d√t (FIG. 35F, G), also showed D-serine concentration dependency (Table 4, FIG. 35H). In the square root of time, OCP decreased immediately with constant linearity (R²≥0.98) for about 2 s in 20 μM to 30 mM D-serine, and the rate of OCP change showed Michaelis-Menten-type dependency against the concentration of D-serine. Remarkably, dOCP/d√t values showed steady value immediately (due to the potentiostat ability in this experiment, the shortest period of the measurement was limited as 100 μs) after the measurement (FIG. 35G, H). From these results, maximum OCP change rate and Kₘ₍ₐₚₚ₎ were calculated by Eadie-Hofstee plot as −33.1±2.4 mV/s^{1/2} and 1.3±0.05 mM, respectively. From these results, the OCP change with respect to the square root of time (dOCP/d√t), transient potentiometry data, demonstrated a good correlation with the D-serine concentration. Considering the rapid and sensitive response observed in the transient potentiometry based on dOCP/d√t monitoring, for the further investigation, dOCP/d√t was selected as the read-out value of quasi-DET-OCP based D-serine sensor.

TABLE 4
Open circuit potential (OCP) responses at enzyme electrodes immobilized with amine-reactive
phenazine ethosulfate-modified D-amino acid oxidase (DAAOx) Gly52Val (G52V) mutant.
D-Ser conc. (mM)

| | 0 mM | 0.02 mM | 0.05 mM | 0.2 mM | 0.5 mM | 2 mM | 5 mM | 10 mM | 20 mM | 30 mM |
|---|---|---|---|---|---|---|---|---|---|---|
| SLOPE | 0.0 ± | −1.0 ± | −2.2 ± | −4.8 ± | −9.2 ± | −17.2 ± | −24.4 ± | −29.7 ± | −33.4 ± | −34.4 ± |
| | 0.2 | 0.1 | 0.1 | 0.4 | 0.9 | 1.4 | 2.0 | 2.4 | 2.5 | 2.4 |
| Y-INTERCEPT | 99.8 ± | 99.6 ± | 99.5 ± | 98.8 ± | 97.8 ± | 96.3 ± | 94.4 ± | 92.7 ± | 91.4 ± | 90.9 ± |
| | 0.1 | 0.0 | 0.0 | 0.1 | 0.8 | 0.3 | 0.5 | 0.8 | 0.9 | 1.0 |
| R² | 0.03 ± | 0.98 ± | 1.00 ± | 1.00 ± | 1.00 ± | 1.00 ± | 1.00 ± | 1.00 ± | 1.00 ± | 1.00 ± |
| | 0.65 | 0.00 | 0.00 | 0.00 | 0.00 | 0.00 | 0.00 | 0.00 | 0.00 | 0.00 |

The slope indicates the OCP change rate against square root of time, y-intercept, and correlation coefficient (R²) for the correlation between the OCP and square root of time within 2 s after the measurement. Each value is presented as the average of the results of three independent electrodes (n=3) and their standard deviations. A potentiostat (SP-150) was used for the experiments.

Continuous operation of DET-type transient potentiometry based D-serine sensor

In order to develop a D-serine biosensing system using the dOCP/d√t, a protocol was developed for intermittent observation of OCP changes. In this protocol, D-serine concentration can be measured continuously by repeating the application of a 100 mV vs. Ag/AgCl potential for 100 μs, followed by OCP monitoring for 1.9 s, which is the interval where OCP decreases linearly with respect to the square root of time in FIG. 35G. Using this protocol, continuous OCP measurement was performed with a PES-modified DAAOx G52V immobilized electrode, while sequentially adding D-serine to the measurement buffer to observe the OCP change with the addition of D-serine.

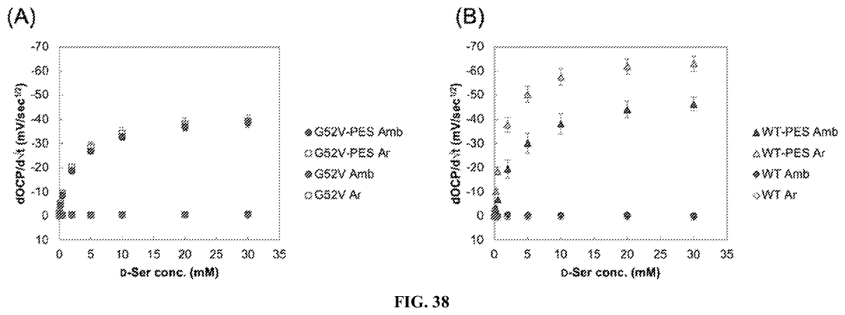

FIGS. 37A and 37B show a time course of OCP in this continuous OCP measurement. In this result, OCP was obviously changed upon the addition of D-serine in the solution, which was indicated by black arrows. Represented OCP change at each concentration of D-serine were summarized in FIG. 37C. D-serine concentration dependency of dOCP/d√t was confirmed for continuous operation protocol of OCP monitoring. The LOD was 20 μM, and its linear range (R² of dOCP/d√t vs. D-serine concentration≥0.99) was from 20 μM to 0.5 mM, with a sensitivity of −16.8 mV/s^{1/2}/mM in PES-modified DAAOx G52V electrode both under ambient conditions and under Ar atmosphere, as are shown in FIG. 38A.

To investigate the impact of oxygen on the sensor response, similar measurements were carried out under argon gas atmosphere, and under ambient air condition, using electrodes with PES-modified DAAOx G52V and with PES-modified DAAOx wild type (WT). PES-modified DAAOx G52V-immobilized electrode, revealed to show identical sensor response and calibration curves for D-serine concentration, under both conditions (FIG. 38A). A 3σ limit of detection (LOD) toward D-serine was 20 μM for both of ambient and argon gas atmosphere for PES-modified DAAOx G52V-immobilized electrode. On the other hand, no OCP change were observed for the PES-unmodified DAAOx G52V-immobilized electrode (FIG. 38A).

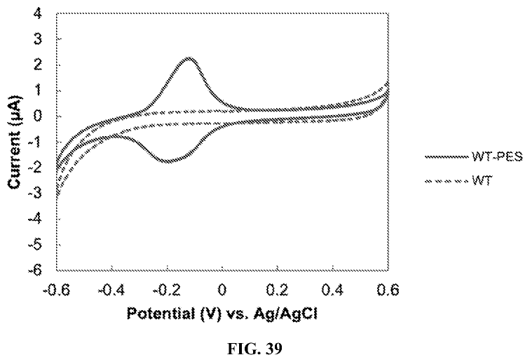

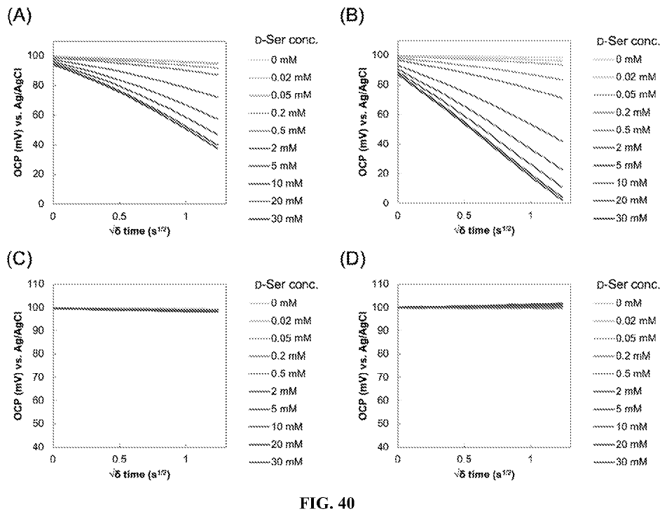

In order to verify the effect of DAAOx G52V with reduced reactivity to oxygen, similar experiments were also performed for DAAOx WT. The on-site modification of the DAAOx WT-immobilized electrode via NTA-SAM and Ni ion by arPES was performed, and successful PES modification was confirmed, the same as DAAOx G52V (FIG. 39). PES-modified DAAOx WT-immobilized electrode showed D-serine concentration dependent dOCP/d√t increase under argon gas atmosphere. However, under ambient atmosphere conditions (FIG. 38B), significant decrease in dOCP/d√t was observed. The maximum dOCP/d√t (dOCP/d√tₘₐₓ) and Kₘ₍ₐₚₚ₎ for D-serine were calculated for DAAOx WT-PES and G52V-PES electrodes in ambient and argon gas atmosphere conditions (Table 5). The dOCP/d√tₘₐₓ of DAAOx WT-PES was −47.2±4.9 mV/s^{1/2} and −64.7±3.5 mV/s^{1/2} under ambient and argon gas atmosphere conditions, respectively, and clearly increased in argon gas atmosphere. In contrast, the DAAOx G52V-PES electrodes showed almost identical values of −39.2±2.0 mV/s^{1/2} and −39.9±2.7 mV/s^{1/2}, respectively. For Kₘ₍ₐₚₚ₎, only the ambient atmosphere condition of DAAOx WT-PES showed a higher value of 3.5±0.7 mM, while the other three conditions were almost identical (argon gas atmosphere of DAAOx WT-PES; 1.3±0.1 mM, ambient atmosphere of DAAOx G52V-PES; 1.9±2.0 mM, and argon gas atmosphere of DAAOx G52V-PES; 1.7±0.01 mM) which were close to 1.8±0.3 mM and 1.4±0.4 mM as the values obtained in the dye-mediated dehydrogenase activity assay.

TABLE 5
Sensor characteristics of amine-reactive phenazine
ethosulfate (PES) - modified D-amino acid oxidase (DAAOx)
wild-type (WT) and Gly52Val (G52V) mutant immobilized
enzyme electrodes under ambient and argon gas atmosphere.

| | Ambient air | | Ar gas | |
|---|---|---|---|---|
| | dOCP/ | | dOCP/ | |
| | d√tₘₐₓ (mV/s^{1/2}) | Kₘ₍ₐₚₚ₎ (mM) | d√tₘₐₓ (mV/s^{1/2}) | Kₘ₍ₐₚₚ₎ (mM) |
| WT-PES | −47.2 ± 4.9 | 3.5 ± 0.7 | −64.7 ± 3.5 | 1.3 ± 0.1 |
| G52V-PES | −39.2 ± 2.0 | 1.9 ± 0.1 | −39.9 ± 2.7 | 1.7 ± 0.01 |

The continuous open circuit potential (OCP) measurement was performed by repeating the procedure: applying a potential of 100 mV vs. AgAgCl for 0.1 s and the OCP measurement for 1.9 s. Maximum OCP change rates (dOCP/d√tₘₐₓ) and apparent affinities of sensor to D-serine (Kₘ₍ₐₚₚ₎) were calculated from the obtained OCP change rate (against the square root of time) at each D-serine concentration. The measurements were carried out on three independent electrodes (n=3) under each condition, and each parameter is shown as an average with standard deviations. A potentiostat (SP-150) was used for the experiments.

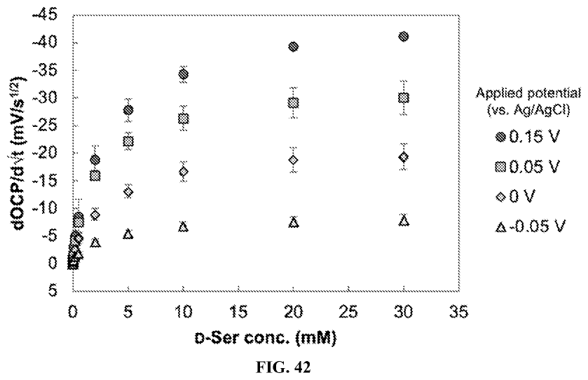

The specificity of D-serine sensor with PES-modified DAAOx G52V electrode was tested by observing its response to three different amino acids: L-serine, an isomer of D-serine; L-glutamate, a major amino acid-type neurotransmitter and D-aspartate; a D-amino acid that exists in the mammalian brain along with D-serine. Almost no response to these three amino acids was observed (FIG. 42). These results suggest that D-serine can be specifically monitored without the influence of dissolved oxygen or other amino acids by using an enzyme electrode of DAAOx G52V modified with PES, which removes only oxidase activity but maintains dehydrogenase activity, in combination with the transient potentiometry based measurement principle.

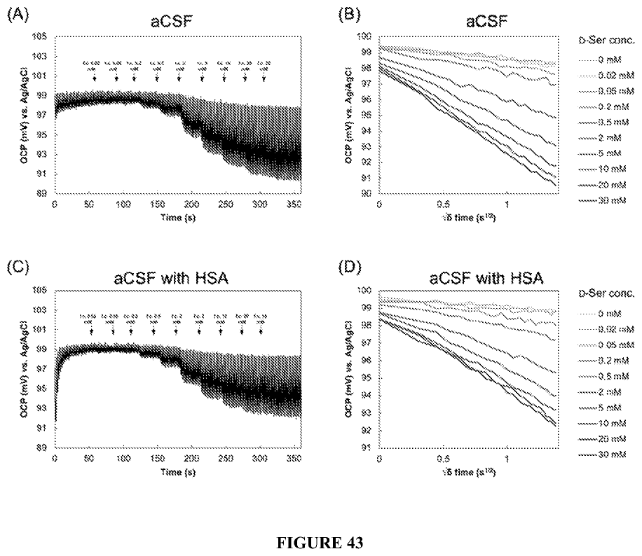

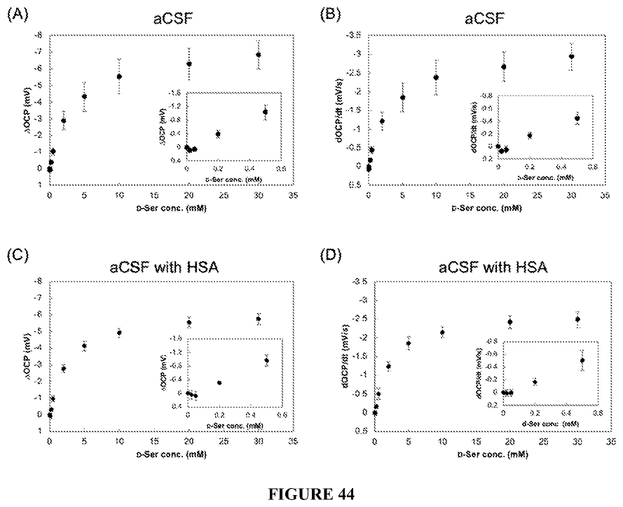

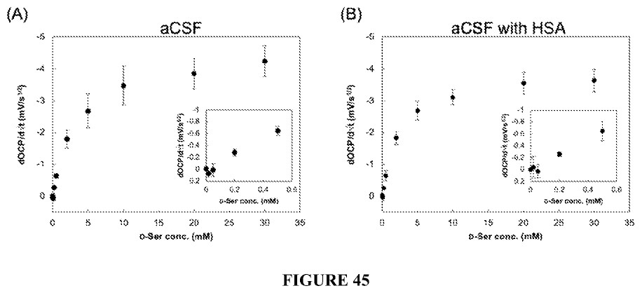

To evaluate OCP measurements in a complex biological environment, we performed D-serine measurements using artificially prepared cerebrospinal fluid (aCSF). Referring to previous papers on the measurement of D-serine using aCSF, aCSF was prepared which mimics the composition of each ion and pH in human CSF (Polcari et al., 2014; Moussa et al., 2021), and measured D-serine using the continuous OCP measurement protocol discussed hereinabove (continuously by repeating the application of a 100 mV vs. Ag/AgCl potential for 100 μs, followed by OCP monitoring for 1.9 s). The results showed that even in aCSF, OCP changed in response to sequential addition of D-serine (FIG. 43A), and it changed linearly with the square root of time (FIG. 43B). In further evaluation of the effect of protein components as well as ionic strength, a modified aCSF (aCSF with HSA) that mimics the protein concentration in human CSF using human albumin, which is the most abundant protein in human CSF, was also evaluated in the same manner, referring to Schirinzi et al., 2021. The OCP measurements in this aCSF with HSA showed the same results toward D-serine as the OCP response in aCSF without protein (FIGS. 43C and 43D). This evaluation showed that D-serine measurement based on the OCP measurement principle using the PES-modified DAAOx G52V electrode developed in this study is possible even in complex aCSFs, and it is expected that D-serine measurement is possible in a medium such as natural CSF and extracellular fluid of the brain. These D-serine concentration-dependent OCP changes were analyzed in the same way as before, and the calibration curves for ΔOCP (FIGS. 44A and 44C), dOCP/dt (FIGS. 44B and 44D), and dOCP/d√t (FIGS. 45A and 45B) against D-serine concentration were shown. As a result, similar calibration was obtained for all analysis methods, and all 3a LODs were 0.2 mM.

### DISCUSSION

In this study, an electrochemical continuous D-serine biosensing system was developed using a quasi-DET-type DAAOx, immobilized by Ni-NTA-SAM by employing the OCP measurement principle, and transient potentiometry using dOCP/d√t value was proposed as the measure for rapid and sensitive D-serine monitoring.

D-serine is a D-amino acid that is associated with major glutamatergic neurotransmission in the mammalian brain. It binds to the glycine modulatory site on the GluN1 side of the N-methyl-D-aspartate receptor (NMDAR) which is particularly localized at excitatory synapses, and thereby regulates ion influx by the NMDAR through L-glutamate binding in neurotransmissions (Uno and Coyle, 2019). Therefore, in order to observe executive D-serine control in neurotransmission, it is necessary to achieve D-serine monitoring in the synaptic cleft where the NMDAR is localized. On the other hand, the brain contains several amino acids such as L-glutamate, L-serine and D-aspartate. Therefore, D-serine measurement requires D-serine-specific, continuous, real-time, in vivo measurements in a heterogeneous environment with such a variety of amino acids. Amperometric biosensors for D-serine monitoring using DAAOx WT have been developed (Pernot et al., 2008; Pernot et al., 2012; Polcari et al., 2014; Polcari et al., 2017; Perry et al., 2018; Campos-Beltrán et al., 2018). However, the effect of dissolved oxygen could not be prevented due to its principle, and the development of a measurement system that does not require oxygen has been desired. In this situation, the G52V mutation in DAAOx was considered to be useful, as stop-flow evaluation had showed that the reactivity to oxygen is greatly reduced, while maintaining the reductive half-reactivity (Saam et al., 2010). Since these evaluations were performed mainly from spectroscopic stop-flow analysis based on the absorption of FAD using D-alanine, though, it was unclear whether D-serine could be used or whether it could donate electrons to an artificial electron acceptor (mediator). Therefore, the oxidase activity and the dye-mediated dehydrogenase activity was evaluated using artificial electron acceptors (mediators), and the results confirmed that DAAOx G52V remains the ability to donate electrons to artificial synthesize electron acceptors (mediators) other than oxygen (FIG. 32).

In terms of oxidase activity, DAAOx WT showed an activity of 7.0±0.4 U/mg at 30 mM D-serine, whereas DAAOx G52V showed maximumly 0.06±0.01 U/mg, indicating that the oxidase activity was 113-fold decreased by the G52V mutation. This value was in agreement with a previous study (Saam et al., 2010), which reported that the reactivity of free, reduced DAAOx G52V with oxygen is lowered ≈100-fold compared to DAAOx WT. The dye-mediated dehydrogenase activities of DAAOx WT showed lower activity than DAAOx G52V at less than 20 mM of D-serine due to competition between oxygen and the artificial electron acceptor (mediator) in DAAOx WT. The same tendency was observed in previous study in reduction of reactivity to oxygen in fructosyl amino acid oxidases (EC 1.5.3) (Kim et al., 2010) and L-lactate oxidase (EC 1.1.3.2) (Hiraka et al., 2018). While the same activities were observed at 30 mM, it was considered due to the diffusion limitation of the artificial electron acceptor (mediator) in DAAOx G52V. In the electrochemical OCP measurement, the reactivity of the PES-modified DAAOx WT electrode was clearly reduced in argon gas atmosphere, while the PES-modified DAAOx G52V electrode showed comparable reactivity because the effect of dissolved oxygen was greatly reduced by G52V mutation (FIG. 38). This is the first report of DAAOx-based D-serine biosensing that is not affected by dissolved oxygen.

There are only a few DET-type enzymes capable of use in enzyme sensors (e.g., cellobiose dehydrogenase (CDH) (EC1.1.99.18), glucose dehydrogenase (GDH) (EC 1.1.5.9), flavocytochrome b2 (Fcb2) (EC 1.1.2.3)) and attempts have been made to design and engineer novel artificial DET-type enzyme to enable measurement of a wide range of target. One of these strategies is genetically fusing the electron-transfer domain derived from DET-type enzyme into the enzyme, directly (Ito et al., 2019: Ito et al., 2021; Hiraka et al., 2021). However, in order to apply these approaches, the structure of a DET-type enzyme that is similar to that of the target enzyme is required, which makes it difficult to adopt DAAOx for this study. Therefore, an enzyme was designed with quasi-DET capability by directly modifying the enzyme with an artificial electron acceptor (mediator) instead of the electron transfer domain from the DET-type enzyme as reported previously (Hatada et al. 2018). In this approach, the lysine residues on the enzyme surface are chemically modified with artificial synthetic electron acceptors (or mediators), and quasi-DET through the modified artificial synthetic electron acceptors (or mediators) can be observed. However, when the same operation was performed in previous studies, the enzyme formed aggregates and the active water-soluble enzyme could not be recovered.

To solve this problem, on-site modification of the DAAOx with arPES after immobilization on the Au electrode was performed as described in Takamatsu et al., 2021. Even with this modification method, quasi-DET currents were observed (FIG. 34), whereas the number of modified PESs and the effect of PES modification on the enzyme could not be evaluated in detail. However, in previous studies on the development of quasi-DET-type enzymes by arPES modification, the DET activity was artificially enhanced by introducing a lysine residue near the active center of the enzyme and arranging the modification site of arPES (Suzuki et al. 2020; Hiraka et al. 2020; Hatada et al. 2021). By applying the same method to DAAOx G52V, it is expected to improve the efficiency of electron transfer between FAD and PES and to measure D-serine with high sensitivity.

In this study, it was attempted to develop a biosensing system by combining a novel quasi-DET enzyme and OCP measurement principle. The OCP changing with the concentration of D-serine was observed at the PES-modified DAAOx immobilized electrode, and as described in section theory, linear (R²≥0.98 for 20 μM to 30 mM D-serine) changes of OCP depending on the square root of time were observed (Table 4). However, the OCP change rate (the slope of the OCP change vs. the square root of time) gradually decayed with time under the high concentration of D-serine condition, and reached a plateau around −120 mV vs Ag/AgCl. From these observations, it is assumed that the decrease of OCP due to the progress of enzymatic reaction and the auto-oxidation of electrode due to such as reduction of oxygen occur on electrode simultaneously and gradually settle down to the equilibrium state. This OCP change due to both local anodic and cathodic reactions, or mixed OCP, has been also reported in (Percival et al., 2017; Baez et al., 2020). Similar to the previous study of OCP measurement using DET-type GDH (Lee et al., 2019), the changing of steady-state OCP from initial OCP value (ΔOCP) also showed D-serine concentration dependence (FIG. 35B). However, the time to reach the OCP at steady-state was much longer than that of DET-type GDH, and the response curve depended on the sampling time within 200 s (FIG. 35B and Table 2), and it was difficult to realize continuous measurement of D-serine using this steady state OCP. On the other hand, the dOCP/d√t considered to reflect the activity of the immobilized enzyme, and as long as the enzyme activity is constant, the OCP change proceeds at a constant rate with respect to the square root of time. In fact, the slope of the dOCP/d√t was constant with respect to the square root of time (FIGS. 35G and 37C). Therefore, the D-serine measurement independent of sampling time was achieved by using this parameter.

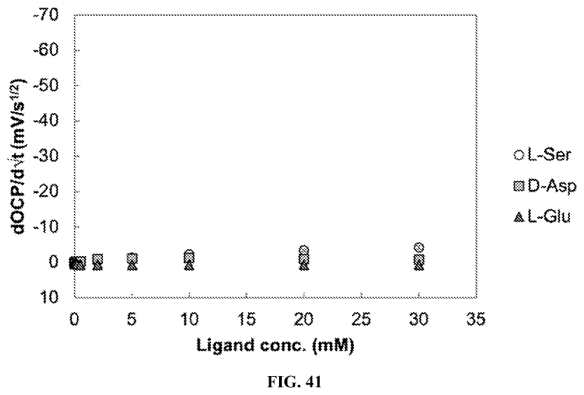

For achieving continuous OCP-based monitoring, the repeating of potential application of 100 mV vs. Ag/AgCl for 100 μs followed by OCP measurement was performed. In an examination of applied potential, the OCP response of a sensor was decreased with decreasing applied potential for applied potentials below approximately 100 mV vs. Ag/AgCl (FIG. 41 and Table 6), while the difference between 150 mV vs. Ag/AgCl and 100 mV vs. Ag/AgCl (FIGS. 38A-B and Table 5) was small. The initial amount of oxidized PES produced, i.e., the amount of PES that can be reduced by the enzyme, was changed by varying the applied potential, and as described in the theory section, the OCP responsiveness was accordingly changed. Currently, potential application of 100 mV vs. Ag/AgCl for 100 μs was sufficient to monitor the D-serine, but in order to achieve higher time-resolution (μ-second order), a period of potential application must be reduced as well, and proportionally the applied potential to be increased to achieve sufficient sensitivity. Meanwhile, the 100 mV vs. Ag/AgCl was included in the redox potential of ascorbic acid, which is a substance commonly mentioned in electrochemical sensors, and monoamines such as dopamine and serotonin, which are present in large amounts in the mammalian brain. The effects of these interferences were not evaluated in this study, but if these effects are significant, they can be addressed by lowering the applied potential to around 0 mV vs. Ag/AgCl and extending the applied time.

TABLE 6
Evaluation of applied potential for continuous open circuit
potential (OCP) measurement using amine-reactive phenazine
ethosulfate (PES)-modified D-amino acid oxidase
(DAAOx) Gly52Val (G52V) mutant immobilized electrode.

| | dOCP/ | |
|---|---|---|
| Applied potential | d√tₘₐₓ (mV/sec^{1/2}) | Kₘ₍ₐₚₚ₎ (mM) |
| 150 mV | −41.1 ± 4.7 | 2.1 ± 1.5 |
| 50 mV | −30.6 ± 3.2 | 1.6 ± 0.2 |
| 0 mV | −19.2 ± 1.3 | 1.7 ± 0.2 |
| −50 mV | −7.9 ± 0.9 | 1.7 ± 0.2 |

The continuous measurement of OCP was performed by repeating the two steps of applying various potentials (150, 50, 0 and −50 mV) vs. AgAgCl potential for 0.1 s and measuring OCP for 1.9 s. From the obtained OCP change rate as shone in FIG. S7, the maximum OCP change rate (dOCP/d√tₘ) and the of the sensor to D-serine (Kₘ₍ₐₚₚ₎) were calculated by Eadie-Hofstee plot. Each value shown is the average of triplicate results (n=3) and the standard deviation.

### CONCLUSION

The development of transient potentiometry based D-serine sensor employing quasi-DET-type enzyme electrode is reported, for future application to neurological disease monitoring. A R. gracilis-derived DAAOx G52V mutant was revealed to possess dye-mediated dehydrogenase activity using artificial synthetic electron acceptors, while its oxidase activity was negligible. DAAOx G52V was immobilized on an Au electrode and was modified with PES, resulting in an enzyme electrode showing quasi-DET type response. Although OCP based monitoring took more than several minutes to obtain steady state OCP value, the time dependent OCP change monitoring, transient potentiometry, provided rapid and sensitive sensor signals. While dOCP/dt based monitoring was suitable for sensing with longer than 5 seconds time resolution with D-serine concentration range between 0.5 mM-5 mM, dOCP/d√t based monitoring is suitable for D-serine monitoring with much shorter time resolution (less than 1 sec) with high sensitivity with wider dynamic range (20 μM-30 mM). dOCP/d√t showed the Michaelis-Menten-type substrate dependence. The maximum dOCP/d√t was −39.2±2.0 mV/s^{1/2}, the Kₘ₍ₐₚₚ₎ was 1.9 mM, and the lower limit of detection was 20 μM. The biosensor developed in this study combines the engineering of the DAAOx mutant into a quasi-DET-type enzyme with a novel OCP measurement principle, which does not require oxygen or addition of external electron acceptors for its measurement, and can be used in continuous, real-time, in vivo, monitoring at nanoscale.

Many modifications and other embodiments of the inventions set forth herein will come to mind to one skilled in the art to which the inventions pertain having the benefit of the teachings presented in the foregoing descriptions and the associated drawings. Therefore, it is to be understood that the inventions are not to be limited to the specific embodiments disclosed and that modifications and other embodiments are intended to be included within the scope of the appended claims. Although specific terms are employed herein, they are used in a generic and descriptive sense only and not for purposes of limitation. Each embodiment disclosed herein is contemplated as being applicable to each of the other disclosed embodiments. All combinations and sub-combinations of the various elements described herein are within the scope of the embodiments.

### LIST OF CITED REFERENCES

Andersen, K. R., Leksa, N. C., & Schwartz, T. U., 2013. Proteins, 81(11), 1857-1861.
Baez, J. F., Compton, M., Chahrati, S., Cánovas, R., Blondeau, P., & Andrade, F. J., 2020. Anal. Chim. Acta, 1097, 204-213.
Campos-Beltrán, D., Konradsson-Geuken, Å., Quintero, J. E., & Marshall, L., 2018. Biosensors, 8(1), 20.
Clausmeyer, J., Schuhmann, W., 2016. TrAC Trend. Anal. Chem., 79, 46-59.
Guercio, G. D., & Panizzutti, R., 2018. Front. Psychiatry., 9, 14.
Hatada, M., Loew, N., Inose-Takahashi, Y., Okuda-Shimazaki, J., Tsugawa, W., Mulchandani, A., Sode, K., 2018. Bioelectrochem., 121, 185-190.
Hatada, M., Saito, S., Yonehara, S., Tsugawa, W., Asano, R., Ikebukuro, K., & Sode, K., 2021. Biosens. Bioelectron., 177, 112984.
Hiraka, K., Kojima, K., Lin, C. E., Tsugawa, W., Asano, R., La Belle, J. T., & Sode, K., 2018. Biosens. Bioelectron., 103, 163-170.
Hiraka, K., Kojima, K., Tsugawa, W., Asano, R., Ikebukuro, K., & Sode, K., 2020. Biosens. Bioelectron., 151, 111974.
Hiraka, K., Tsugawa, W., Asano, R., Yokus, M. A., Ikebukuro, K., Daniele, M. A., & Sode, K., 2021. Biosens. Bioelectron., 176, 112933.
Ito, K., Okuda-Shimazaki, J., Kojima, K., Mori, K., Tsugawa, W., Asano, R., . . . & Sode, K., 2021. Biosens. Bioelectron., 176, 112911.
Ito, K., Okuda-Shimazaki, J., Mori, K., Kojima, K., Tsugawa, W., Ikebukuro, K., . . . & Sode, K., 2019. Biosens. Bioelectron., 123, 114-123.
Ito, Y., Okuda-Shimazaki, J., Tsugawa, W., Loew, N., Shitanda, I., Lin, C. E., . . . & Sode, K., 2019. Biosens. Bioelectron., 129, 189-197.
Katz, E., Bückmann, A. F., & Willner, I., 2001. J. Am. Chem. Soc., 123(43), 10752-10753.
Kim, S., Nibe, E., Ferri, S., Tsugawa, W., & Sode, K., 2010. Biotechnol. Lett., 32(8), 1123-1129.
Lee, D., & Cui, T., 2012. Microelectron. Eng., 93, 39-42.
Lee, I., Loew, N., Tsugawa, W., Ikebukuro, K., Sode, K., 2019. Biosens. Bioelectron., 124, 216-223.
MacKay, M. A. B., Kravtsenyuk, M., Thomas, R., Mitchell, N. D., Dursun, S. M., Baker, G. B., 2019. Front. Psychiatry, 10, 25.
Madeira, C., Lourenco, M. V., Vargas-Lopes, C., Suemoto, C. K., Brandão, C. O., Reis, T., . . . Grinberg, L. T., 2015. Transl. Psychia., 5, e561-e561.
Moussa, S., Van Horn, M. R., Shah, A., Pollegioni, L., & Thibodeaux, C. J., 2021, J. Electrochem. Soc., 168(2), 025502
Percival, S. J., Bard, A. J., 2017. Anal. Chem., 89(18), 9843-9849.
Pernot, P., Maucler, C., Tholance, Y., Vasylieva, N., Debilly, G., Pollegioni, L., . . . & Marinesco, S., 2012. Neurochem. Int., 60(8), 837-845.
Pernot, P., Mothet, J. P., Schuvailo, O., Soldatkin, A., Pollegioni, L., Pilone, M., . . . & Marinesco, S., 2008. Anal. Chem., 80(5), 1589-1597.
Perry, S. C., Gateman, S. M., Sifakis, J., Pollegioni, L., & Mauzeroll, J., 2018. J. Electrochem. Soc. 165(12), G3074.
Pilone, M. S., 2000. Cell. Mol. Life Sci., 57(12), 1732-1747.
Polcari, D., Kwan, A., Van Horn, M. R., Danis, L., Pollegioni, L., Ruthazer, E. S., & Mauzeroll, J., 2014. Anal. Chem., 86(7), 3501-3507.
Polcari, D., Perry, S. C., Pollegioni, L., Geissler, M., & Mauzeroll, J., 2017. ChemElectroChem, 4(4), 920.
Rassas, I., Braiek, M., Bonhomme, A., Bessueille, F., Raffin, G., Majdoub, H., & Jaffrezic-Renault, N., 2019. Sensors, 19(1), 154.
Rosini, E., Molla, G., Ghisla, S., & Pollegioni, L., 2011. FEBS J., 278(3), 482-492.
Saam, J., Rosini, E., Molla, G., Schulten, K., Pollegioni, L., & Ghisla, S., 2010. J. Biol. Chem., 285(32), 24439-24446.
Schirinzi, T., Cordella, A., Mercuri, N. B., D'Amico, A . . . & Santonico, M., 2021, Sensors (Basel), 21(11), 3767
Smith, L. A., Glasscott, M. W., Vannoy, K. J., Dick, J. E., 2019. Anal. Chem., 92, 2266-2273.
Sonia, F., Loredano, P., & Mirella, P. S., 2001. Enzyme Microb. Tech., 29(6-7), 407-412.
Studier, F. W., 2005. Protein Expr. Purif, 41(1), 207-234.
Suzuki, N., Lee, J., Loew, N., Takahashi-Inose, Y., Okuda-Shimazaki, J., Kojima, K., . . . & Sode, K., 2020. Int. J. Mol. Sci., 21(3), 1137.
Takamatsu, S., Lee, J., Asano, R., Tsugawa, W., Ikebukuro, K., & Sode, K., 2021. Sens. Actuators B: Chem., 346, 130554.
Uno, Y., & Coyle, J. T., 2019. Psych. Clin. Neurosci., 73(5), 204-215.
Zain, Z. M., O'Neill, R. D., Lowry, J. P., Pierce, K. W., Tricklebank, M., Dewa, A., & Ab Ghani, S., 2010. Biosen. Bioelectron., 25(6), 1454-1459.

What is claimed is:

1. A method of measuring a target substance concentration in a sample comprising:
contacting the sample comprising the target substance with a biosensor which comprises an enzyme electrode comprising an oxidoreductase immobilized on the electrode, and a reference electrode;
measuring a time-dependent change of an open circuit potential between the enzyme electrode and the reference electrode; and
calculating the concentration of the target substance based on the time-dependent change of the open circuit potential.

2. The method of claim 1, wherein the biosensor further comprises a counter electrode.

3. The method of claim 1, wherein the target substance is selected from the group consisting of D-serine, lactate, glucose, glycated proteins, glycated amino acid, hydrogen peroxide, cholesterol, glycerol, glycerol-3-phosphate, fructose, urate, ethanol, galactose, 1,5-anhydro-D-glucitol, NAD(P)H, dopamine, 3-hydroxybutyrate, Levodopa (L-DOPA), L-glutamate, L-glutamine, sarcosine, creatine, and creatinine.

4. The method of claim 3, wherein the target substance is selected from the group consisting of D-serine, lactate, glucose, glycated proteins, glycated amino acid, hydrogen peroxide, cholesterol, glycerol, glycerol-3-phosphate, fructose, urate, ethanol, galactose, 1,5-anhydro-D-glucitol, NAD(P)H, dopamine, 3-hydroxybutyrate, and Levodopa (L-DOPA.

5. The method of claim 1, wherein the measuring is continuous.

6. The method of claim 1, wherein no potential is applied before measuring the time-dependent change of the open circuit potential.

7. The method of claim 1, wherein the time for an initial measurement is less than 60 seconds.

8. The method of claim 7, wherein the time for an initial measurement is less than about 1 second.

9. The method of claim 1, wherein the oxidoreductase is selected from the group consisting of oxidases, dehydrogenases, monooxigenases, and dioxygenases.

10. The method of claim 1, wherein the oxidoreductase is selected from the group consisting of glucose dehydrogenase, glucose oxidase, lactate oxidase, lactate dehydrogenase, D-amino acid oxidase, fructosyl amino acid/peptide oxidases, peroxidase, cholesterol oxidase, glycerol-3-phosphate oxidase, cellobiose dehydrogenase, and fructose dehydrogenase, uricase, alcohol oxidase, alcohol dehydrogenase, galactose oxidase, galactose dehydrogenase, pyranose oxidase, pyranose dehydrogenase, glucose-3-dehydrogenase, diaphorase, thyrosinase, 3-hydroxybutyrate dehydrogenase, amine oxidase, monoamine oxidase, polyamine oxidase, dopamine β-monooxygenase, 4,5-DOPA dioxygenase extradiol, glutamate oxidase, and sarcosine oxidase.

11. The method of claim 10, wherein the oxidoreductase is selected from the group consisting of glucose dehydrogenase, glucose oxidase, lactate oxidase, lactate dehydrogenase, D-amino acid oxidase, fructosyl amino acid/peptide oxidases, peroxidase, cholesterol oxidase, glycerol-3-phosphate oxidase, cellobiose dehydrogenase, and fructose dehydrogenase, uricase, alcohol oxidase, alcohol dehydrogenase, galactose oxidase, galactose dehydrogenase, pyranose oxidase, pyranose dehydrogenase, glucose-3-dehydrogenase, diaphorase, thyrosinase, 3-hydroxybutyrate dehydrogenase, amine oxidase, monoamine oxidase, polyamine oxidase, dopamine β-monooxygenase, and 4,5-DOPA dioxygenase extradiol.

12. The method of claim 1, wherein the oxidoreductase is an engineered oxidoreductase.

13. The method of claim 12, wherein the engineered oxidoreductase is a fusion enzyme.

14. The method of claim 3, wherein the target substance is glucose and the oxidoreductase is glucose dehydrogenase.

15. The method of claim 3, wherein the target substance is glucose and the oxidoreductase is glucose oxidase.

16. The method of claim 3, wherein the target substance is lactate and the oxidoreductase is lactate oxidase.

17. The method of claim 3, wherein the target substance is D-serine and the oxidoreductase is D-amino acid oxidase.

18. The method of claim 3, wherein the enzyme electrode, counter electrode, or reference electrode is less than about 100 μm in diameter.

19. The method of claim 18, wherein the enzyme electrode, counter electrode, or reference electrode is less than about 10 μm in diameter.

20. The method of claim 19, wherein the enzyme electrode, counter electrode, or reference electrode is less than about 1 μm.

21. The method of claim 1, wherein the reference electrode is a leakless reference electrode.

22. The method of claim 1, wherein the sample is a biological sample.

23. The method of claim 1, wherein the electrode is at least partially in organic solvent.

24. The method of claim 1, wherein the time-dependent change is dOCP/dt.

25. The method of claim 1, wherein the time-dependent change is dOCP/d√t.

26. A biosensor for measuring a concentration of a target substance in a sample comprising an enzyme electrode comprising an oxidoreductase immobilized on the electrode and a reference electrode.

27. The biosensor of claim 26, further comprising a counter electrode.

28. The biosensor of claim 26, wherein the reference electrode is a leakless reference electrode comprising a sealed platinum wire.

29. The biosensor of claim 27, wherein the enzyme electrode, counter electrode, or reference electrode is less than about 2 mm in diameter.

30. The biosensor of claim 26, wherein the oxidoreductase is selected from the group consisting of oxidases, dehydrogenases, monooxigenases, and dioxygenases.

31. The biosensor of claim 26, wherein the oxidoreductase is selected from the group consisting of glucose dehydrogenase, glucose oxidase, lactate oxidase, lactate dehydrogenase, D-amino acid oxidase, fructosyl amino acid/peptide oxidases, peroxidase, cholesterol oxidase, glycerol-3-phosphate oxidase, cellobiose dehydrogenase, and fructose dehydrogenase, uricase, alcohol oxidase, alcohol dehydrogenase, galactose oxidase, galactose dehydrogenase, pyranose oxidase, pyranose dehydrogenase, glucose-3-dehydrogenase, diaphorase, thyrosinase, 3-hydroxybutyrate dehydrogenase, amine oxidase, monoamine oxidase, polyamine oxidase, dopamine β-monooxygenase, 4,5-DOPA dioxygenase extradiol, glutamate oxidase, and sarcosine oxidase.

32. The biosensor of claim 31, wherein the oxidoreductase is selected from the group consisting of glucose dehydrogenase, glucose oxidase, lactate oxidase, lactate dehydrogenase, D-amino acid oxidase, fructosyl amino acid/peptide oxidases, peroxidase, cholesterol oxidase, glycerol-3-phosphate oxidase, cellobiose dehydrogenase, and fructose dehydrogenase, uricase, alcohol oxidase, alcohol dehydrogenase, galactose oxidase, galactose dehydrogenase, pyranose oxidase, pyranose dehydrogenase, glucose-3-dehydrogenase, diaphorase, thyrosinase, 3-hydroxybutyrate dehydrogenase, amine oxidase, monoamine oxidase, polyamine oxidase, dopamine β-monooxygenase, and 4,5-DOPA dioxygenase extradiol.

33. The biosensor of claim 26, wherein the target substance is selected from the group consisting of D-serine, lactate, glucose, glycated proteins, glycated amino acid, hydrogen peroxide, cholesterol, glycerol, glycerol-3-phosphate, fructose, urate, ethanol, galactose, 1,5-anhydro-D-glucitol, NAD(P)H, dopamine, 3-hydroxybutyrate, Levodopa (L-DOPA), L-glutamate, L-glutamine, sarcosine, creatine, and creatinine.

34. The biosensor of claim 33, wherein the target substance is selected from the group consisting of D-serine, lactate, glucose, glycated proteins, glycated amino acid, hydrogen peroxide, cholesterol, glycerol, glycerol-3-phosphate, fructose, urate, ethanol, galactose, 1,5-anhydro-D-glucitol, NAD(P)H, dopamine, 3-hydroxybutyrate, and Levodopa (L-DOPA).

35. The biosensor of claim 31, wherein the target substance is glucose and the oxidoreductase is glucose dehydrogenase.

36. The biosensor of claim 31, wherein the target substance is glucose and the oxidoreductase is glucose oxidase.

37. The biosensor of claim 31, wherein the target substance is lactate and the oxidoreductase is lactate oxidase.

38. The biosensor of claim 31, wherein the target substance is D-serine and the oxidoreductase is D-amino acid oxidase.

39. The biosensor of claim 26, wherein the oxidoreductase is an engineered oxidoreductase.

40. The biosensor of claim 26, wherein the oxidoreductase is an engineered oxidoreductase, the reference electrode is a leakless reference electrode comprising a sealed platinum wire, and the enzyme electrode and reference electrode are each less than about 100 μm in diameter.

41. The biosensor of claim 40, wherein the enzyme electrode or reference electrode is less than about 10 μm in diameter.

42. The biosensor of claim 40, wherein the enzyme electrode or reference electrode is less than about 1 μm.
